SEQUENCE LISTING (16 sequences)

SEQ ID NO: 1 - DNA, 4011 bp, Homo sapiens
gggtagagacggggtttcaccgtgttagccaggatggtctggatctcctgacctcgtgatccacccacctcggcctcctaaagtgctgggattacagacatgagccaccgcgcccagccctattcatcccttttcaaaagtcagaccctaggaagctggagggaggtggggcatggttttacagtgaatttctgatttcactcagggtgataaatcagactcttggggaagcgggtggtggctctggacagcagcaggaatggggatccagttagcaacaaatccatggacctatgacaggctgaaagccaccccttctccatctttgggaggttgccaatgtctgatttaacactatccaatgaatgatcattgaaagtaaaaaataactatcaactagcagaaaatataaatggtaagcattagcacatatttcacatgtttatatttggctctcagattgacctataaaacaaagtctgggaaattctatatgatcctgaaaaaatgatacgctggtctggatggtagaataagttggagaaatgtttaagccaaaatgcagtcttaccaatgactttttattttattttattaattttcaggatttttggtatacaggtggtttttggttacatggaaaagttctttactggtgatttctgagattttagttcaccccttatcctgagcagtgtacactgttcccaatatgtagccttttatccctcaccccctctaagttcaagaagactatggtcctgcagaaagctttatatgtaattaacatatctttatctttatctttataggcagtagactcatcttttgaaacagattccattaagagtgaatgtgtaccctccctctagcctttattattactgtttttgctattacatgtgttagtgtatgtgaatttaatgcttaaaaatgtatcccattggctactatggcaaaaggttgactcataagagtttagcacgggttaagatctgaaagttttctcccagcctcttatcactggcgcagacttcacaattcatggaagccaccagtgagatgacattgcctcaggcagttactatttttatattctataactcgaggagctcagggtttcggaaatcattaaactttttttgtccttttaaagttggagacagcaattgtagacagccttccagtgggttatctttttgtgtctccttacctgtggagaagcctattagctgggatatgtagttaaatagctatatttatatatatccagggcaccccgaattcgggagagcttcccggagtcgaccttcctgctggctgctctgtgaccgcttcccggctctgccctcttggccgaagtgcccgctgccgggcgcgggcctcagacaatacaatggtgggtgaagagaagatgtctctaagaaaccggctgtcaaagtccagggaaaatcctgaggaagatgaagaccagagaaaccctgcaaaggagtccctagagacacctagtaatggtcgaattgacataaaacagttgatagcaaagaagataaagttgacagcagaggcagaggaattgaagccattttttatgaaggaagttggcagtcactttgatgattttgtgaccaatctcattgaaaagtcagcatcattagataatggtgggtgcgctctcacaaccttttctgttcttgaaggagagaaaaacaaccatagagcgaaggatttgagagcacctccagaacaaggaaagatttttattgcaaggcgctctctcttagatgaactgcttgaagtggaccacatcagaacaatatatcacatgtttattgccctcctcattctctttatcctcagcacacttgtagtagattacattgatgaaggaaggctggtgcttgagttcagcctcctgtcttatgcttttggcaaatttcctaccgttgtttggacctggtggatcatgttcctgtctacattttcagttccctattttctgtttcaacattgggccactggctatagcaagagttctcatccgctgatccgttctctcttccatggctttcttttcatgatcttccagattggagttctaggttttggaccaacatatgttgtgttagcatatacactgccaccagcttcccggttcatcattatattcgagcagattcgttttgtaatgaaggcccactcatttgtcagagagaacgtgcctcgggtactaaattcagctaaggagaaatcaagcactgttccaatacctacagtcaaccagtatttgtacttcttatttgctcctacccttatctaccgtgacagctatcccaggaatcccactgtaagatggggttatgtcgctatgaagtttgcacaggtctttggttgctttttctatgtgtactacatctttgaaaggctttgtgcccccttgtttcggaatatcaaacaggagcccttcagcgctcgtgttctggtcctatgtgtatttaactccatcttgccaggtgtgctgattctcttccttactttttttgcctttttgcactgctggctcaatgcctttgctgagatgttacgctttggtgacaggatgttctataaggattggtggaactccacgtcatactccaactattatagaacctggaatgtggtggtccatgactggctatattactatgcttacaaggactttctctggtttttctccaagagattcaaatctgctgccatgttagctgtctttgctgtatctgctgtagtacacgaatatgccttggctgtttgcttgagctttttctatcccgtgctcttcgtgctcttcatgttctttggaatggctttcaacttcattgtcaatgatagtcggaaaaagccgatttggaatgttctgatgtggacttctcttttcttgggcaatggagtcttactctgcttttattctcaagaatggtatgcacgtcggcactgtcctctgaaaaatcccacatttttggattatgtccggccacgttcctggacttgtcgttacgtgttttagaagcttggactttgtttcctccttgtcactgaagattgggtagctccctgatttggagccagctgtttccagttgttactgaagttatctgtgttatttggaccactccaggctttacagatgactcactccattcctaggtcacttgaagccaaactgttggaagttcactggagtcttgtacacttaagcagagcagaactttttttgtggggctgggtggggggagaagaccgactaacagctgaagtaatgacagattgttgctgggtcatatcagctttatcccttggtaattatatctgttttgtttcttgactctgtccaatcagagaataaacatcatagtttcttggccactgaattagccaaaacacttaggaagaaatcacttaaatacctctggcttagaaattttttcatgcacactgttggaatgtatgctaattgaacatgcaattggggaagaaaaaatgtagaatgatttttgctatttctagtagaaagaaaatgtctgttttccaaagataatgttatacatcctattttgtaatttttttgaaaaaagttcaatgttcagttttccttagtttttaccttgttttctctataggtcatgatttctgtgaagcaaaaagatgccttttaccatgaattcttgagtttacatcaataatattgtatattaaggggatcagaagtaggaaggaaaaaataagagatagcagaggaaaaagaaaaacatttcctcttataacttctgaagtaatttgtaaaaaagatttgtagagtcaatcatgtgtttaaattattttatcacaaacttaacatggaagatattcctttttaactttgtggtaacttctttgaagttatttagaaatatcctttggaacaattattttattgtctaataaatattgacttctcttgaattattttgcagactagtgagtctgtac

SEQ ID NO: 2 - protein, 550 aa, Homo sapiens
MVGEEKMSLRNRLSKSRENPEEDEDQRNPAKESLETPSNGRIDIKQLIAKKIKLTAEAEELKPFFMKEVGSHFDDFVTNLIEKSASLDNGGCALTTFSVLEGEKNNHRAKDLRAPPEQGKIFIARRSLLDELLEVDHIRTIYHMFIALLILFILSTLVVDYIDEGRLVLEFSLLSYAFGKFPTVVWTWWIMFLSTFSVPYFLFQHWATGYSKSSHPLIRSLFHGFLFMIFQIGVLGFGPTYVVLAYTLPPASRFIIIFEQIRFVMKAHSFVRENVPRVLNSAKEKSSTVPIPTVNQYLYFLFAPTLIYRDSYPRNPTVRWGYVAMKFAQVFGCFFYVYYIFERLCAPLFRNIKQEPFSARVLVLCVFNSILPGVLILFLTFFAFLHCWLNAFAEMLRFGDRMFYKDWWNSTSYSNYYRTWNVVVHDWLYYYAYKDFLWFFSKRFKSAAMLAVFAVSAVVHEYALAVCLSFFYPVLFVLFMFFGMAFNFIVNDSRKKPIWNVLMWTSLFLGNGVLLCFYSQEWYARRHCPLKNPTFLDYVRPRSWTCRYVF

SEQ ID NO: 3 - DNA, 2040 bp, Homo sapiens
agaagaggcaacacgggcaagggctgcctgctgcccgctggagaccgcaccatggagccaggcggggcccgtctgcgtctgcagaggacagaagggctgggaggggagcgggagcgccaaccctgtggagatggaaacactgagacgcacagagccccggacttggtacaatggacccgacacatggaggctgtgaaggcacaattgctggagcaagcgcagggacaactgagggagctgctggatcgggccatgcgggaggctatacaatcctacccatcacaagacaaacctctgcccccacctcccccaggttccttgagcaggacccaggagccatccctggggaaacagaaagttttcatcatccgcaagtccctgcttgatgagctgatggaggtgcagcatttccgcaccatctaccacatgttcatcgctggcctgtgtgtcttcatcatcagcaccctggccatcgacttcattgatgagggcaggctgctgctggagtttgacctactgatcttcagcttcggacagctgccattggcgctggtgacctgggtgcccatgtttctgtccaccctgttggcgccgtaccaggccctacggctgtgggccaggggcacctggacgcaggcgacgggcctgggctgtgcgctgctagccgcccacgccgtggtgctctgcgcgctgccggtccacgtggccgtggagcatcagctcccgccggcctcccgttgtgtcctggtcttcgagcaggttaggttcctgatgaaaagctactccttcctgagagaggctgtgcctgggacccttcgtgccagacgaggtgaggggatccaggcccccagtttctccagctacctctacttcctcttctgcccaacactcatctacagggagacttaccctaggacgccctatgtcaggtggaattatgtggccaagaactttgcccaggccctgggatgtgtgctctatgcctgcttcatcctgggccgcctctgtgttcctgtctttgccaacatgagccgagagcccttcagcacccgtgccctggtgctctctatcctgcatgccacgttgccaggcatcttcatgctgctgctcatcttctttgccttcctccattgctggctcaacgcctttgccgagatgctacgatttggagacaggatgttctaccgggactggtggaactcaacgtccttctccaactactaccgcacttggaacgtggtggtccatgactggctgtacagctacgtgtatcaggatgggctgcggctccttggtgcccgggcccgaggggtagccatgctgggtgtgttcctggtctccgcagtggcccatgagtatatcttctgcttcgtcctggggttcttctatcccgtcatgctgatactcttccttgtcattggaggaatgttgaacttcatgatgcatgaccagcgcaccggcccggcatggaacgtgctgatgtggaccatgctgtttctaggccagggaatccaggtcagcctgtactgccaggagtggtacgcacggcggcactgccccttaccccaggcaactttctgggggctggtgacacctcgatcttggtcctgccatacctagaggtcgggacagacgacgctacctgcccagacaccaccaagttctctgcctgcaaaacctggggaccaggactccctgtctgcattccccaaatttggctctgagtcgaggcaacctgcacacaagacccccacccaaggaatgtgcaaggactgagatctgcagacttgtgggtaactgatcacagacctcagcatgggggtgaccagggtgactcttcaatccctatccccatgggctgggtacaggatatcctcctaccccatgactgtcttagggagacttggggtaccttatggatttgatgaatgtgggggaactcagaggaactggggccaccaaggttggaaaagggtttggttcttgactttgtattccttccaatacagcaataaactttgtctccctttttattcattc

SEQ ID NO: 4 - protein, 522 aa, Homo sapiens
MEPGGARLRLQRTEGLGGERERQPCGDGNTETHRAPDLVQWTRHMEAVKAQLLEQAQGQLRELLDRAMREAIQSYPSQDKPLPPPPPGSLSRTQEPSLGKQKVFIIRKSLLDELMEVQHFRTIYHMFIAGLCVFIISTLAIDFIDEGRLLLEFDLLIFSFGQLPLALVTWVPMFLSTLLAPYQALRLWARGTWTQATGLGCALLAAHAVVLCALPVHVAVEHQLPPASRCVLVFEQVRFLMKSYSFLREAVPGTLRARRGEGIQAPSFSSYLYFLFCPTLIYRETYPRTPYVRWNYVAKNFAQALGCVLYACFILGRLCVPVFANMSREPFSTRALVLSILHATLPGIFMLLLIFFAFLHCWLNAFAEMLRFGDRMFYRDWWNSTSFSNYYRTWNVVVHDWLYSYVYQDGLRLLGARARGVAMLGVFLVSAVAHEYIFCFVLGFFYPVMLILFLVIGGMLNFMMHDQRTGPAWNVLMWTMLFLGQGIQVSLYCQEWYARRHCPLPQATFWGLVTPRSWSCHT

SEQ ID NO: 5 - DNA, 22 bp, Artificial Sequence
  oligonucleotide primer
tccaggtgtgttcagtagttcc

SEQ ID NO: 6 - DNA, 22 bp, Artificial Sequence
  oligonucleotide primer
gaagccatctctgaggtctgtg

SEQ ID NO: 7 - DNA, 19 bp, Artificial Sequence
  oligonucleotide primer
agcctgtctgtgattgctg

SEQ ID NO: 8 - DNA, 20 bp, Artificial Sequence
  oligonucleotide primer
agatgaccgtaagcaccacc

SEQ ID NO: 9 - DNA, 23 bp, Artificial Sequence
  oligonucleotide primer
agagctacagttaccactcttcg

SEQ ID NO: 10 - DNA, 23 bp, Artificial Sequence
  oligonucleotide primer
gaggtccatgctaaacttgacaa

SEQ ID NO: 11 - DNA, 20 bp, Artificial Sequence
  oligonucleotide primer
gtggtgctgctcttcctcat

SEQ ID NO: 12 - DNA, 20 bp, Artificial Sequence
  oligonucleotide primer
ttggggctgttcctcatctg

SEQ ID NO: 13 - DNA, 19 bp, Artificial Sequence
  oligonucleotide primer
caagtatgagacagtgccc

SEQ ID NO: 14 - DNA, 21 bp, Artificial Sequence
  oligonucleotide primer
gccatctatttcccaactctg

SEQ ID NO: 15 - DNA, 22 bp, Artificial Sequence
  oligonucleotide primer
ttacgagagaaaactcatggcc

SEQ ID NO: 16 - DNA, 22 bp, Artificial Sequence
  oligonucleotide primer
gggtccaagttgtccagaatgc

(19) United States
(12) Patent Application Publication
(10) Pub. No.: US 2015/0087649 A1
(43) Pub. Date: Mar. 26, 2015

(54) TREATING DISORDERS ASSOCIATED WITH ABERRANT ADRENOCORTICAL CELL BEHAVIOR

(71) Applicant: Atterocor, Inc., Ann Arbor, MI (US)

(72) Inventors: Stephen Warren Hunt, III, Dexter, MI (US); Julia Christine Owens, Ann Arbor, MI (US); William E. Rainey, JR., Ann Arbor, MI (US); Christopher R. LaPensee, Ann Arbor, MI (US); Gary D. Hammer, Ann Arbor, MI (US)

(21) Appl. No.: 14/498,849

(22) Filed: Sep. 26, 2014

Related U.S. Application Data
(60) Provisional application No. 61/883,089, filed on Sep. 26, 2013.
(60) Provisional application No. 62/015,187, filed on Jun. 20, 2014.

(51) Int. Cl. A61K 31/5375; A61K 31/27; A61K 31/167; A61K 31/343; A61K 31/4436; A61K 31/216; A61K 31/4178

(52) U.S. Cl. CPC A61K 31/5375; A61K 31/216; A61K 31/27; A61K 31/4178; A61K 31/343; A61K 31/4436; A61K 31/167
USPC 514/237.8; 514/539; 514/476; 514/397; 514/469; 514/336; 514/630

(57) ABSTRACT

Methods and agents are provided for treatment of disorders associated with aberrant adrenocortical cell behavior, including (but not limited to) treatment of adrenocortical carcinoma (ACC) and/or Cushing's syndrome. Such methods involve administration of an agent which exhibits an IC₅₀ value against huACAT1 of less than 10 μM, and one or more further characteristics including effects on adrenocortical cells, disruption of cholesterol homeostasis, reduction in steroid biosynthesis, reduction of mitochondrial function, and/or preferential binding to by low-density lipoprotein (LDL).

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 120205_{—}403_SEQUENCE_LISTING.txt. The text file is 18.4 KB, was created on Sep. 26, 2014, and is being submitted electronically via EFS-Web.

### BACKGROUND

1. Technical Field

Methods and agents are provided for treatment of disorders associated with aberrant adrenocortical cell behavior.

2. Description of the Related Art

The adrenal gland is made up of two parts: the outer cortex in which certain hormones are produced, and the inner medulla which is part of the nervous system, wherein nervous system hormones are produced. The cortex is devoted to the synthesis of glucocorticoid, mineralocorticoid and androgen hormones. Specific cortical cells produce particular hormones including aldosterone, cortisol, and androgens such as androstenedione. Adrenocortical tumors originate in the cortex.

There are two main types of adrenal cortex tumors: adenomas which are benign and adrenocortical carcinoma which are malignant. Adenomas in many people produce no symptoms, but in some instances the tumors lead to excess hormone production. Adrenocortical carcinoma can produce the hormones cortisol, aldosterone, estrogen, or testosterone, as well as other hormones. Adrenocortical carcinomas (ACC) are rare, highly malignant tumors. In women, the tumor often releases these hormones, which can lead to male characteristics. The excess hormones may or may not cause symptoms. In general, adenomas are treated by removal of the adrenal gland or with therapeutic intervention. Likewise, adrenocortical carcinomas can lead to hormone production that can cause noticeable body changes such as weight gain, fluid build-up, early puberty in children, or excess facial or body hair in women. While the cause is unknown, adrenocortical carcinoma is most common in children younger than 5 and adults, on average, in their 40s. Adrenocortical carcinoma may be linked to a cancer syndrome that is passed down through families (inherited). Both men and women can develop this tumor.

While the understanding of the disease has advanced with the advent of modern molecular techniques, the prognosis of patients with advanced disease, who represent about half of the diagnoses, remains dismal. Targeted therapies are in clinical development, but whether they will yield breakthroughs in the management of the disease is yet unknown (Hammer, G. D. and T. Else, eds., Adrenocortical Carcinoma, Basic Science and Clinical Concepts, 2011, New York: Springer).

The sole FDA-approved therapeutic agent for ACC is mitotane (o,p′-DDD), a derivative of the insecticide DDT, discovered in 1950s, when it was found to destroy the adrenal cortex of dogs. Despite half a century of use, its molecular mechanism remains unclear. The drug requires chemical transformation into an active, free radical form, which then induces lipid peroxidation and cell death. Mitotane also suppresses steroidogenesis and inhibits other cytochrome P450-class enzymes (Id.).

Whereas mitotane is widely used for the treatment of ACC, it has increased progression-free survival in only one-quarter to one-third of patients. For the patients that derive a therapeutic benefit, the effect is transient, delaying disease progression by an average of five months (Id.). Mitotane has numerous problems as a therapeutic agent, making its use difficult, and requiring close monitoring of patients.

Accordingly, there remains a significant need for new therapeutic agents useful for treatment of ACC and other related diseases or conditions. One such promising agent is N-(2,6-bis(1-methylethyl)phenyl)-N′-((1-(4-(dimethylamino)phenyl)cyclopentyl)-methyl)urea hydrochloride (“ATR-101”).

While advances have been made in this field, there remains a need in the art for additional methods and agents for treatment of disorders associated with aberrant adrenocortical cell behavior, including but not limited to ACC. The present invention fulfills these needs and provides further related advantages.

### BRIEF SUMMARY

In brief, methods and agents are provided for treatment of disorders associated with aberrant adrenocortical cell behavior.

In one aspect, the present disclosure provides a method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) reduces steroid biosynthesis in adrenocortical cells, and wherein the agent is not N-(2,6-bis(1-methylethyl)phenyl)-N′-((1-(4-(dimethylamino)phenyl)-cyclopentyl)-methyl)urea or a salt thereof.

In certain embodiments, the agent further reduces cholesteryl ester levels in adrenocortical cells compared to untreated adrenocortical cells.

In certain embodiments, the agent reduces cortisol biosynthesis in adrenocortical cells. In certain further aspects, the reduction in cortisol biosynthesis is evaluated by increase in ACTH.

In certain embodiments, the agent reduces DHEA-S biosynthesis in adrenocortical cells.

In certain embodiments, the agent reduces pregnenolone, progesterone, 11-deoxycorticosterone, corticosterone, 18OH-corticosterone, aldosterone, 17OH-pregnenolone, 17OH-progesterone, 11-deoxycortisol, 11-dehydrocorticosterone, cortisone, dehydroepiandrosterone, androstenediol-17α, androstenediol-17β, androstenedione, testosterone, dehydroepiandrosterone sulfate, estrone, or estradiol biosynthesis in adrenocortical cells.

In certain embodiments, the agent exhibits an IC₅₀ value against huACAT1 of less than 5 μM. In further embodiments, the agent exhibits an IC₅₀ value against huACAT1 of less than 3 μM, 1 μM, 0.5 μM, 0.3 μM, 0.1 μM, 0.05 μM, 0.03 μM, 0.01 μM, 0.005 μM, 0.003 μM, or 0.001 μM.

In certain embodiments, the agent is selected from any one of the compounds listed in Table 1.

In another aspect, the present disclosure provides for a method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) reduces mitochondrial function in adrenocortical cells, and wherein the agent is not N-(2,6-bis(1-methylethyl)phenyl)-N′-((1-(4-(dimethylamino)phenyl)-cyclopentyl)-methyl)urea or a salt thereof.

In certain embodiments, the agent has low tissue concentration in the liver compared to the adrenal gland.

In certain embodiments, the agent exhibits an IC₅₀ value against huACAT1 of less than 5 μM. In further embodiments, the agent exhibits an IC₅₀ value against huACAT1 of less than 3 μM, 1 μM, 0.5 μM, 0.3 μM, 0.1 μM, 0.05 μM, 0.03 μM, 0.01 μM, 0.005 μM, 0.003 μM, or 0.001 μM.

In certain embodiments, the agent is selected from any one of the compounds listed in Table 1.

In another aspect, the present disclosure provides a method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) is preferentially bound by low-density lipoprotein (LDL), and wherein the agent is not N-(2,6-bis(1-methylethyl)phenyl)-N′-((1-(4-(dimethylamino)phenyl)-cyclopentyl)-methyl)urea or a salt thereof.

In certain embodiments, the agent is preferentially distributed to the adrenal gland or the ovary.

In certain embodiments, the agent exhibits an IC₅₀ value against huACAT1 of less than 5 μM. In further embodiments, the agent exhibits an IC₅₀ value against huACAT1 of less than 3 μM, 1 μM, 0.5 μM, 0.3 μM, 0.1 μM, 0.05 μM, 0.03 μM, 0.01 μM, 0.005 μM, 0.003 μM, or 0.001 μM.

In certain embodiments, the agent is selected from any one of the compounds listed in Table 1.

In yet another aspect, the present disclosure provides a method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) has effects on one or more adrenocortical cells, and wherein the agent is not N-(2,6-bis(1-methylethyl)phenyl)-N′-((1-(4-(dimethylamino)phenyl)cyclopentyl)-methyl)urea or a salt thereof.

In certain embodiments, the effects on adrenocortical cells are measured by TUNEL staining or activated caspase-3 and/or caspase 7 immunostaining.

In certain embodiments, the agent does not have effects on liver cells.

In certain embodiments, the agent exhibits an IC₅₀ value against huACAT1 of less than 5 μM. In further embodiments, the agent exhibits an IC₅₀ value against huACAT1 of less than 3 μM, 1 μM, 0.5 μM, 0.3 μM, 0.1 μM, 0.05 μM, 0.03 μM, 0.01 μM, 0.005 μM, 0.003 μM, or 0.001 μM.

In yet another aspect, the present disclosure provides a method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) disrupts cholesterol homeostasis in adrenocortical cells, and wherein the agent is not N-(2,6-bis(1-methylethyl)phenyl)-N′-((1-(4-(dimethylamino)phenyl)cyclopentyl)-methyl)urea or a salt thereof.

In certain embodiments, the agent causes an increase in one or more of the following in adrenocortical cells compared to untreated adrenocortical cells: calcium influx, activation of unfolded protein response (UPR), and initiation of apoptosis.

In certain embodiments, the increase in calcium influx is determined by measuring increase in expression of at least one calcium-sensitive gene selected from CYP11B2, MC2R, NR4A2, MRAP, and INHBA.

In certain embodiments, the increase in activation of unfolded protein response is determined by measuring increase in expression of an activated XBP-1 splice variant.

In certain embodiments, the increase in initiation of apoptosis is determined by measuring TUNEL staining or activated caspase-3 and/or caspase 7 immunostaining.

In certain embodiments, the agent exhibits an IC₅₀ value against huACAT1 of less than 5 μM, 3 μM, 1 μM, 0.5 μM, 0.3 μM, 0.1 μM, 0.05 μM, 0.03 μM, 0.01 μM, 0.005 μM, 0.003 μM, or 0.001 μM.

In certain embodiments, the agent is selected from any one of the compounds listed in Table 1.

Additional aspects of the present disclosure provide for methods for treating a disorder associated with aberrant adrenocortical cell behavior according to any one the aforementioned embodiments, wherein the disorder is adrenocortical carcinoma, benign adenoma, increased hormone production, metastatic adrenocortical carcinoma, congenital adrenal hyperplasia, Cushing's syndrome, excess cortisol production, symptoms associated with excess cortisol production, hyperaldosteronism, 21-hydroxylase deficiency, reduces adrenocortical tumor size, or aberrant adrenal hormone production. In certain embodiments, the disorder is adrenocortical carcinoma. In certain embodiments, the disorder is Cushing's syndrome.

### BRIEF DESCRIPTION OF THE DRAWINGS

FIGS. 1A-1C show exemplary transcript and polypeptide sequences for human ACAT1 (SEQ ID NOs:1 and 2, respectively) and ACAT2 (SEQ ID NOs: 3 and 4, respectively).

FIG. 2 shows Caspase-3/7 activity in H295R human adrenocortical cancer cells treated with representative adrenal acting and non-adrenal acting ACAT1 inhibitors in the presence of exogenous cholesterol.

FIG. 3 shows free cholesterol (FC) and esterified cholesterol (CE) in H295R human adrenocortical cancer cells treated with representative adrenal acting and non-adrenal acting ACAT1 inhibitors.

FIG. 4 shows XBP-1 splicing in H295R human adrenocortical cancer cells treated with representative adrenal acting and non-adrenal acting ACAT1 inhibitors in the presence of exogenous cholesterol.

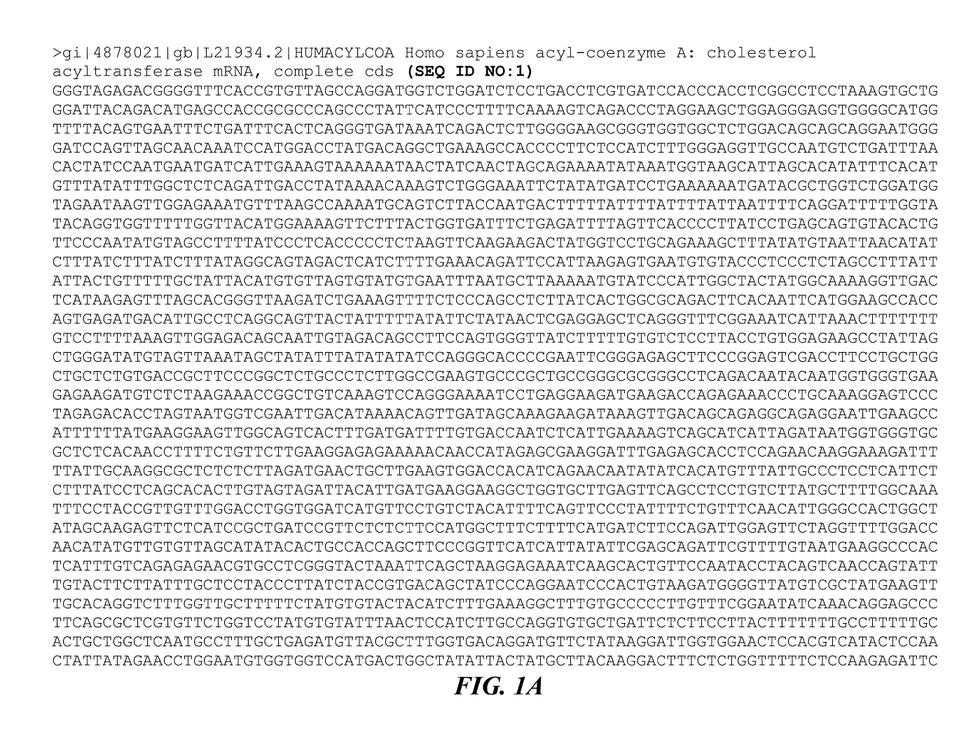

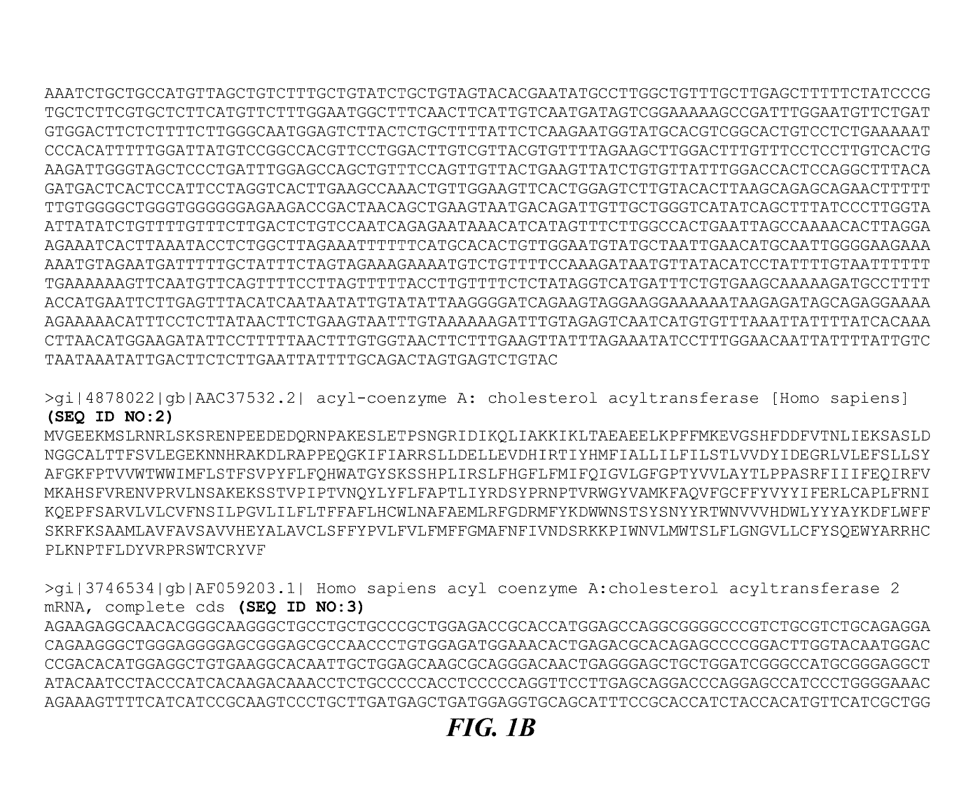

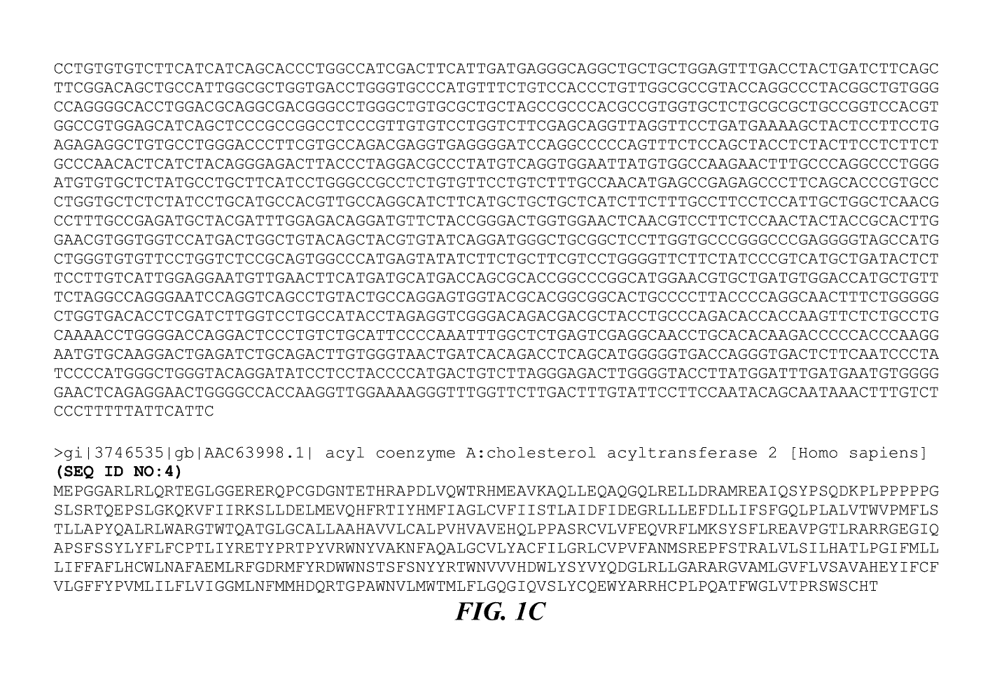

### DETAILED DESCRIPTION

As mentioned above, methods and agents are provided for treatment of disorders associated with aberrant adrenocortical cell behavior. Such methods involve administering to a subject in need of such treatment a therapeutically effective amount of an agent as defined in more detail below.

As used herein, “treatment” includes therapeutic applications to slow or stop progression of a disorder associated with aberrant adrenocortical cellular behavior, prophylactic application to prevent development of a disorder associated with aberrant adrenocortical cellular behavior, and reversal of a disorder associated with aberrant adrenocortical cellular behavior. Reversal of a disorder differs from a therapeutic application which slows or stops a disorder in that with a method of reversing, not only is progression of a disorder completely stopped, cellular behavior is moved to some degree, toward a normal state that would be observed in the absence of aberrant adrenocortical cellular behavior.

As used herein, “aberrant adrenocortical cellular behavior” includes increased hormone production, Cushing's syndrome, benign adenoma, adrenocortical carcinoma, metastatic adrenocortical carcinoma, congenital adrenal hyperplasia, hyperaldosteronism including Conn syndrome, a unilateral aldosterone-producing adenoma, bilateral adrenal hyperplasia (or idiopathic hyperaldosteronism (IHA)), renin-responsive adenoma, primary adrenal hyperplasia and glucocorticoid-remediable aldosteronism (GRA), and 21-hydroxylase deficiency. Accordingly, “disorders associated with aberrant adrenocortical cellular behavior” is used herein to mean symptoms and/or conditions that arise, either directly or indirectly, from aberrant adrenocortical cellular behavior. As will become apparent herein, these symptoms and/or conditions that arise, either directly or indirectly, from aberrant adrenocortical cellular behavior are numerous. As used herein, “adrenocortical” and “adrenal cortex” are intended to mean the same.

As used herein, “Cushing's syndrome” means a hormonal disorder caused by prolonged exposure of the body's tissues to high levels of cortisol. Cushing's syndrome is sometimes referred to as “hypercortisolism” (excess cortisol production). Cushing's syndrome includes various subtypes of the disease, including Cushing's disease, adrenal Cushing's syndrome, and ectopic ACTH syndrome, which are categorized by the cause of hypercortisolism. Cushing's disease, also known as pituitary Cushing's, is caused by a pituitary gland tumor which secretes excessive ACTH, which in turn stimulates the adrenal glands to make more cortisol. Ectopic ACTH syndrome is caused by tumors that arise outside the pituitary gland that can produce ACTH, which stimulates cortisol production. Adrenal Cushing's syndrome is caused by an abnormality of the adrenal gland, usually an adrenal tumor, which causes excess cortisol secretion.

As used herein, the phrase “metastatic cancer” is defined as a cancer that has the potential to, or has begun to, spread to other areas of the body.

As used herein, “subject” means a mammal, including a human.

As used herein, the phrase term “therapeutically effective amount” refers to an amount of a therapeutic agent to treat, ameliorate, or prevent a disease or condition, or to exhibit a detectable therapeutic or preventative effect. The effect is detected by, for example, a reduction in tumor size. The effect is also detected by, for example, chemical markers, steroid levels, or antigen levels. Therapeutic effects also include reduction in physical symptoms, such as decreased body temperature. The precise effective amount for a subject will depend upon the subject's size and health, the nature and extent of the condition, the therapeutics or combination of therapeutics selected for administration, and other variables known to those of skill in the art. The effective amount for a given situation is determined by routine experimentation and is within the judgment of the clinician.

An “agent” means a compound that exhibits the characteristics disclosed herein. The agent itself can be the active form, or the agent can be metabolized upon administration to the subject to yield the active form. Thus, as used herein, the term agent also includes a prodrug. To this end, a “prodrug” is a compound typically having little or no pharmacological activity itself but capable of releasing, for example by hydrolysis or metabolic cleaving of a linkage such as an ester moiety, an active agent upon administration to the subject.

As used herein, an “ACAT inhibitor” means an agent that inhibits or reduces human acyl coenzyme A:cholesterol acyltransferase) (huACAT1) activity. ACAT1, also known as sterol o-acyltransferasel (SOAT1), catalyzes the esterification of free cholesterol into cholesteryl esters. An exemplary nucleotide sequence for ACAT1 is provided by Genbank Accession # L21934.2 (SEQ ID NO:1). An exemplary amino acid sequence for ACAT1 is provided by Genbank Accession # AAC37532.2 (SEQ ID NO:2). An ACAT inhibitor exhibits an IC₅₀ value against huACAT1 of less than 10 μM determined by the fluorescent cell-based assay measuring esterification of NBD-cholesterol in AC29 cells expressing huACAT1 as set forth in Example 1.

As used herein, “adrenocortical cells” means cells derived from the adrenal cortex of a subject. The “adrenal cortex” is situated along the perimeter of the adrenal gland and mediates the stress response through production of mineralocorticoids (e.g., aldosterone) and glucocorticoids (e.g., cortisol). The adrenal cortex is also a secondary site of androgen synthesis. Adrenocortical cells may be derived from any one of the three zones of the adrenal cortex, or any combination thereof,—the zona glomerulosa, zona fasciculata, and zona reticularis.

As used herein, “reduces steroid biosynthesis” in adrenocortical cells means reduction of biosynthesis of steroid hormones from cholesterol by adrenocortical cells, and includes reduction of steroid biosynthesis within the adrenocortical cells and/or reduction of the number of adrenocortical cells producing the steroid. Steroid hormones produced by adrenocortical cells include mineralocorticoids, glucocorticoids, estrogens, progestagens, and androgens. Reduction of steroid biosynthesis in adrenocortical cells may be determined by measuring a decrease in a steroid intermediate or end product, including pregnenolone, progesterone, 11-deoxycorticosterone, corticosterone, 18OH-corticosterone, aldosterone, 17OH-pregnenolone, 17OH-progesterone, 11-deoxycortisol, 11-dehydrocorticosterone, cortisone, dehydroepiandrosterone, androstenediol-17α, androstenediol-17β, androstenedione, testosterone, dehydroepiandrosterone sulfate, estrone, or estradiol biosynthesis. An agent reduces steroid biosynthesis if there is a decrease in steroid levels greater than 10% compared to a vehicle-treated control by the methods set forth in Examples 2 and 3.

As used herein, “reduces cholesterol ester levels” in adrenocortical cells compared to untreated adrenocortical cells means that the level of cholesteryl ester in adrenocortical cells is reduced or decreased as a result of administration of an agent as compared with cholesteryl ester levels in untreated adrenocortical cells as measured by the procedure of Example 4. A 10% decrease in cholesteryl ester levels in adrenocortical cells treated with an agent compared to vehicle treated adrenocortical cells is considered significant.

As used herein, “reduces mitochondrial function” in adrenocortical cells means that mitochondrial function has decreased in adrenocortical cells as a result of administration of an agent. A reduction in mitochondrial function may be the result of mitochondrial damage or a significant decline in number. Reduction of mitochondrial function may result from disruption of mitochondrial DNA replication, inhibition of the electron transport chain, uncoupling of ATP synthesis from electron transport, inhibition of the Krebs cycle, inhibition of mitochondrial transporters, changes in mitochondrial membrane permeability, or decreases in the number of mitochondria per cell. Mitochondrial function is assessed by measuring cellular ATP levels as set forth in Example 5.

As used herein, “preferentially bound by low-density lipoprotein (LDL)” means that an agent is found in plasma at higher concentrations in the LDL fraction as compared to the high density lipoprotein (HDL) and/or very low density lipoprotein (VLDL), fractions by the procedure set forth in Example 6.

As used herein, “has effects on adrenocortical cells” or “adrenal acting” means that an agent induces dysfunction in adrenocortical cells. Conversely, “non-adrenal acting” means an agent that does not induce dysfunction in adrenocortical cells. Adrenocortical cell effects may occur by a variety of mechanisms, including impaired steroidogenesis, toxin activation by CYP-450 enzymes, disruption of normal function and structure by exogenous steroids, and/or DNA damage. Morphological manifestations of adrenocortical cell effects may include cytoplasmic vacuolation, granular degeneration, necrosis, cell lysis, apoptosis, accumulation of lipid bodies, swollen endoplasmic reticulum or mitochondria, atrophy, nodular regeneration, fibrosis, hypertrophy, hyperplasia, and/or ultrastructural lesions of phospholipidosis. While the specific mechanism and/or manifestation may vary as noted above, dysfunction in adrenocortical cells generally results in the loss or reduction of adrenocortical cell activity or viability, including the induction of apoptosis and cell death. To this end, adrenocortical cell effects may be assessed by measuring apoptosis, and more specifically the markers of apoptosis as set forth in Examples 7 and 10. Alternatively, adrenocortical cell effects may be assayed by measuring mechanistic and/or manifestation parameters associated with dysfunction of adrenocortical cells, such as reduction in steroid (e.g., cortisol) biosynthesis, reduction in cholesterol esters, reduction in mitochondrial function, and/or cholesterol homeostasis as set forth in Examples 2-5, 8 and 9.

The present disclosure provides methods and compositions for treating a disorder associated with aberrant adrenocortical cell behavior, including but not limited to adrenocortical carcinoma, benign adenoma, increased hormone production, metastatic adrenocortical carcinoma, congenital adrenal hyperplasia, Cushing's syndrome, excess cortisol production, symptoms associated with excess cortisol production, hyperaldosteronism, 21-hydroxylase deficiency, and aberrant adrenal hormone production in a subject. The present disclosure further provides for the identification of an agent for treating a disorder associated with aberrant adrenocortical cell behavior based on the presence of specific characteristics in the agent.

In one aspect, the present disclosure provides a method for treating a disorder associated with aberrant adrenocortical cell behavior in a subject in need thereof, comprising administering to the subject a therapeutically effective amount of an agent which: (a) exhibits an IC₅₀ value against human ACAT1 (huACAT1) of less than 10 μM; and (b) reduces steroid biosynthesis in adrenocortical cells; and wherein the agent is not N-(2,6-bis(1-methylethyl)-phenyl)-N′-((1-(4-(dimethylamino)phenyl)cyclopentyl)-methyl)urea or a salt thereof.

Acyl-coenzyme A:cholesterol transferase (ACAT) is an integral membrane protein localized in the endoplasmic reticulum. ACAT catalyzes formation of cholesteryl esters (CE) (also known as cholesterol esters) from cholesterol and fatty acyl coenzyme A. Cholesteryl esters are stored as cytoplasmic lipid droplets in the cell. In steroidogenic tissues, such as the adrenal gland, cholesteryl esters act as a cholesterol reservoir for biosynthesis of steroid hormones. In mammals, there are two ACAT isoenzymes, ACAT1 and ACAT2. ACAT2 is expressed in the liver and intestine. ACAT1 expression is more ubiquitous and is present in cells and tissues such as macrophages, adrenal glands, hepatocytes, enterocytes, renal tubule cells, and neurons. ACAT1 is the main isoenzyme in the adrenal gland. The major isoform of ACAT1 is a 50 kDa protein. ACAT1 may also be present as a minor 56 kDa protein.

In the methods described herein, the agent inhibits the ability of huACAT1 to catalyze the esterification of free cholesterol into cholesteryl ester. An agent's inhibitory activity and IC₅₀ is measured using a fluorescent cell-based assay measuring esterification of NBD-cholesterol in AC29 cells expressing huACAT1 as described in Lada et al. (J. Lipid Res. 45:378-386, 2004) (hereby incorporated by reference in its entirety) (see Example 1). AC29 cells lack endogenous ACAT1 activity and are transfected to express human ACAT1. The assay uses 22-[N-(7-nitrobenz-2-oxa-1,3-diazol-4-yl)amino]-23,24-bisnor-5-cholen-3-ol (NBD-cholesterol), a fluorescent sterol analog in which the NBD moiety replaces the terminal segment of the alkyl tail of cholesterol. NBD-cholesterol has been shown to mimic native cholesterol absorption in multiple systems. In a polar environment, NBD-cholesterol is weakly fluorescent. In a nonpolar environment, NBD-cholesterol is strongly fluorescent. The fluorescent property of NBD-cholesterol is used to measure ACAT activity, as cholesterol is a polar lipid and cholesteryl ester is nonpolar. Untransfected AC29 cells or AC29 cells expressing huACAT1 treated with a known ACAT inhibitor can be used to determine background fluorescence due to free-NBD-cholesterol.

The half maximal inhibitory concentration (IC₅₀) is a measure of the effectiveness of an agent in inhibiting a biological or biochemical activity. It is the half maximal (50%) inhibitory concentration of an agent. The agent described herein exhibits an IC50 value against human ACAT1 (huACAT1) of less than 10 μM. In certain embodiments, the agent exhibits an IC50 value against huACAT1 of less than 10 μM, 5 μM, 3 μM, 1 μM, 0.5 μM, 0.3 μM, 0.1 μM, 0.05 μM, 0.03 μM, 0.01 μM, 0.005 μM, 0.003 μM, or 0.001 μM. In certain embodiments, the agent exhibits an IC₅₀ value against huACAT1 from about 0.001 μM to about 10 μM, from about 0.001 μM to about 5 μM, from about 0.001 μM to about 3 μM, from about 0.001 μM to about 1 μM, from about 0.01 μM to about 10 μM, from about 0.01 μM to about 5 μM, from about 0.01 μM to about 3 μM, from about 0.01 μM to about 1 μM, from about 0.1 μM to about 10 μM, from about 0.1 μM to about 5 μM, from about 0.1 μM to about 3 μM, or from about 0.1 μM to about 1 μM.

Cholesterol, which has a 17-carbon steroid nucleus, is the precursor of steroid biosynthesis and is converted into steroid hormone intermediates and end products by cytochrome P450 enzymes in the mitochondria and endoplasmic reticulum. Cholesterol may be derived from multiple sources, including de novo synthesis from acetate; absorption as LDLs or HDLs; or lipid droplets containing cholesterol acetate (a cholesterol ester) within adrenocortical cells, which serve as a cholesterol reservoir for steroid biosynthesis. Enzymatic conversion of cholesterol to pregnenalone by CYP11A1 is the rate limiting step for steroid biosynthesis. After synthesis of pregnenalone, synthesis of progestagens, glucocorticoids, mineralocorticoids, androgens, and estrogens may also occur in adrenocortical cells.

In one embodiment, the agent used in the methods described herein also reduces steroid biosynthesis in adrenocortical cells. A steroid includes end product steroids and steroid intermediates. Steroid hormones include mineralocorticoids, glucocorticoids, estrogens, progestagens, and androgens. Reduction in steroid biosynthesis may be the result of a decrease in CE resulting from inhibition of ACAT activity. Reduction of steroid biosynthesis may be determined by measuring a steroid intermediate or end product by liquid chromatography/mass spectrometry (LC/MS) or gas chromatography/mass spectrometry (GC/MS).

In another embodiment, an agent may increase steroid biosynthesis in adrenocortical cells. An agent may also inhibit another enzyme in the steroid biosynthetic pathway, which may reduce steroid intermediates and products downstream of the point of inhibition and/or increase the substrates upstream of the point of inhibition. A change of greater than 10% in one or more steroid intermediates or end products either an increase in an upstream substrate or a decrease in a downstream product as a result of a block at a specific step in steroid biosynthesis—as compared to a vehicle treated control is considered to be significant.

In certain embodiments, an agent changes (increases or decreases) steroid biosynthesis in adrenocortical cells by greater than 10%, 15%, 20%, 25%, 30%, 35%, 40%, 45%, 50%, 55%, 60%, 65%, 70%, 75%, 80%, 85%, 90%, or 95% compared to a vehicle treated control.

In certain embodiments, the agent reduces glucocorticoid biosynthesis in adrenocortical cells. A glucocorticoid may be 11-deoxycorticosterone, corticosterone, 18OH-corticosterone, 11-deoxycortisol, 11-dehydrocorticosterone, cortisol, or cortisone. In a specific embodiment, the agent reduces cortisol biosynthesis in adrenocortical cells. Reduction of cortisol biosynthesis is determined by measuring cortisol levels as set forth in Example 2 or Example 3.

Alternatively, a reduction of cortisol biosynthesis may be evaluated by measuring an increase in ACTH. Glucocorticoid synthesis is regulated by the hypothalamic-pituitary-adrenal (HPA) axis via adrenocorticotropic hormone (ACTH). ACTH increases production of glucocorticoids (e.g., cortisol) via short term and long term effects. ACTH stimulates delivery of cholesterol to the mitochondria, where CYP11A1 cleaves the side chain of cholesterol in the rate limiting step for glucocorticoid biosynthesis. ACTH also stimulates lipoprotein uptake into adrenocortical cells and induces transcription of steroidogenic enzymes. Cortisol production provides a negative feedback to inhibit release of ACTH. Without wishing to be bound by theory, an agent's therapeutic effect on aberrant adrenocortical cell behavior may be associated with increases in serum ACTH levels resulting from disruption of cortisol biosynthesis. ACTH levels are determined as set forth in Example 3.

In certain embodiments, the agent reduces mineralocorticoid biosynthesis in adrenocortical cells. A mineralocorticoid may be aldosterone, 11-deoxycorticosterone, or corticosterone.

In certain embodiments, the agent reduces progestagen biosynthesis in adrenocortical cells. A progestagen may be pregnenolone, progesterone, 17OH-pregnenolone, or 17OH-progesterone.

In certain embodiments, the agent reduces estrogen biosynthesis in adrenocortical cells. An estrogen may be estrone or estradiol.

In certain embodiments, the agent reduces androgen biosynthesis in adrenocortical cells. An androgen may be dehydroepiandrosterone (DHEA), dehydroepiandrosterone sulfate (DHEA-S), androstenedione, androstenediol, testosterone, or dihydrotestosterone. In a specific embodiment, the agent reduces DHEA-S biosynthesis in adrenocortical cells.

In certain embodiments, the agent increases glucocorticoid biosynthesis in adrenocortical cells. A glucocorticoid may be 11-deoxycorticosterone, corticosterone, 18OH-corticosterone, 11-deoxycortisol, 11-dehydrocorticosterone, cortisol, or cortisone. In a specific embodiment, the agent increases cortisol biosynthesis in adrenocortical cells. Increase in cortisol biosynthesis is determined by measuring cortisol levels as set forth in Example 2 or Example 3.

In certain embodiments, the agent increases mineralocorticoid biosynthesis in adrenocortical cells. A mineralocorticoid may be aldosterone, 11-deoxycorticosterone, or corticosterone.

In certain embodiments, the agent increases progestagen biosynthesis in adrenocortical cells. A progestagen may be pregnenolone, progesterone, 17OH-pregnenolone, or 17OH-progesterone.

In certain embodiments, the agent increases estrogen biosynthesis in adrenocortical cells. An estrogen may be estrone or estradiol.

In certain embodiments, the agent increases androgen biosynthesis in adrenocortical cells. An androgen may be dehydroepiandrosterone (DHEA), dehydroepiandrosterone sulfate (DHEA-S), androstenedione, androstenediol, testosterone, or dihydrotestosterone. In a specific embodiment, the agent increases DHEA-S biosynthesis in adrenocortical cells.

In certain embodiments, the agent further reduces cholesteryl ester levels in adrenocortical cells compared to untreated adrenocortical cells. Reduction of cholesteryl ester levels is consistent with the activity of an agent as an ACAT inhibitor with sufficient distribution to adrenocortical cells. Cholesteryl ester levels may be measured by determining total cholesterol and free cholesterol levels as described in Example 4 (see, Can et al., 1993, Clin. Biochem. 26:39-42; hereby incorporated by reference in its entirety). Briefly, lipid extracts are prepared from adrenal glands of treated subjects, and enzymatic assays are used to determine total cholesterol and free cholesterol. Cholesteryl ester is determined by subtracting free cholesterol from total cholesterol. A 10% decrease in the amount of cholesteryl ester in adrenal glands from a treated subject compared to a vehicle-treated control is considered significant.

In certain embodiments, the agent reduces of cholesteryl ester levels in adrenocortical cells by greater than 10%, 15%, 20%, 25%, 30%, 35%, 40%, 45%, 50%, 55%, 60%, 65%, 70%, 75%, 80%, 85%, 90%, or 95% compared to untreated adrenocortical cells.

In certain embodiments, the agent may be any one of the compounds selected from Table 1.

In another aspect, the present disclosure provides a method for treating a disorder associated with aberrant adrenocortical cell behavior in a subject in need thereof, comprising administering to the subject a therapeutically effective amount of an agent which: (a) exhibits an IC₅₀ value against human ACAT1 (huACAT1) of less than 10 μM; and (b) disrupts cholesterol homeostasis in adrenocortical cells; and wherein the agent is not N-(2,6-bis(1-methylethyl)-phenyl)-N′-((1-(4-(dimethylamino)phenyl)cyclopentyl)-methyl)urea or a salt thereof. The agent used in the methods described herein disrupts cholesterol homeostasis in adrenocortical cells. Cholesterol is a critical component for most mammalian cells. Cholesterol is contained in most cell membranes and is important for regulating membrane fluidity. Moreover, cholesterol, as a component of cell membranes, is involved in intracellular transport and cell signaling. As a result, changes in the levels of free cholesterol and/or cholesterol ester, in both macro- and microenvironments, may have significant effects on the physiology and health of the cell.

Inhibition of ACAT1 by the agent leads to an increase in the levels of both substrates of the reaction catalyzed by this enzyme, free cholesterol and fatty acids, which are used to form cholesterol esters. Disruption of cholesterol homeostasis may result in a compromise of membrane integrity that may lead to acute changes, including calcium influx and membrane depolarization, and chronic changes resulting in activation of the unfolded protein response (UPR) and initiation of the apoptotic cascade and subsequent cell death. In a particular embodiment, an agent may cause an increase in one, two, or all of the following responses in adrenocortical cells: calcium influx, activation of the unfolded protein response (UPR), and initiation of apoptosis.

Calcium influx may result in the short term effect of up- or down-regulation of calcium-sensitive genes, and the long term effect of induction of markers of programmed cell death. Fatty acids are members of the sterol family, many of which are ligands for transcriptional regulators and hence are involved in regulation of sensitive genes. Examples of calcium regulated genes include CYP11B2 (steroid 11/18 β hydroxylase), MC2R (melanocortin 2 (ACTH) receptor), NR4A2 (Nuclear Receptor Subfamily 4 Group A Member 2 or Nurr1), MRAP (MC2R accessory protein), and INHBA (inhibin beta A). Calcium influx may be determined by measuring changes in expression of calcium sensitive genes using methods known in the art. In certain embodiments, an agent's effect on cholesterol homeostasis is assessed as set forth in Example 8.

For example, H295R human adrenocortical cancer cells may be plated at 500,000 cells/well in a 12 well plate. After overnight incubation at 37° C., media is removed and replaced with treatment media containing vehicle or the test agent (e.g., an ACAT1 inhibitor at its ACAT1 IC₅₀ concentration) in the presence of exogenous cholesterol. After treatment of cells for various lengths of time (minutes to hours), RNA is extracted using the Qiagen RNEasy Plus kit and 1 μg is reverse transcribed using Applied Biosystems High Capacity RT Kit. Quantitative real-time PCR is performed on cDNA. Target genes are amplified using gene-specific primers (e.g., 5′-TCCAGGTGTGTTCAGTAGTTCC-3′ (SEQ ID NO:5) and 5′-GAAGCCATCTCTGAGGTCTGTG-3′ (SEQ ID NO:6) for CYP11B2; 5′-AGCCTGTCTGTGATTGCTG-3′ (SEQ ID NO:7) and 5′-AGATGACCGTAAGCACCACC-3′ (SEQ ID NO:8) for MC2R; 5′-AGAGCTACAGTTACCACTCTTCG-3′ (SEQ ID NO:9) and 5′-GAGGTCCATGCTAA ACTTGACAA-3′ (SEQ ID NO:10) for NR4A2 (Nurr1); 5′-GTGGTGCTGCTCTTCCTCAT-3′ (SEQ ID NO:11) and 5′-TTGGGGCTGTTCCTCATCTG-3′ (SEQ ID NO:12) for MRAP; and 5′-CAAGTATGAGACAGTGCCC-3′ (SEQ ID NO:13) and 5′-GCCATCTATTTCCCAACTCTG-3′ (SEQ ID NO:14) for INHBA) by qPCR under the following conditions: 1) 90° for 30 s; 2) 95° for 15 s; and 3) 60° for 60 s. Steps 2 through 4 are then repeated for 40 cycles. Gene expression may be normalized to Gapdh, and expressed as fold-change compared to vehicle treated cells, where the control is normalized to 1. Changes in expression of calcium sensitive genes that are at least 2-fold up or down compared to untreated cells are considered to be significant.

In certain embodiments, an agent modifies expression of at least one calcium sensitive gene selected from CYP11B2, MC2R, NR4A2, MRAP, and INHBA. In certain embodiments, an agent modifies expression of at least two, three, four, or all of the calcium sensitive genes selected from CYP11B2, MC2R, NR4A2, MRAP, and INHBA. In certain embodiments, the agent increases expression of one, two, three, four, or all of the calcium sensitive genes selected from CYP11B2, MC2R, NR4A2, MRAP, and INHBA.

In certain embodiments, an agent modifies expression of at least one calcium sensitive gene at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared to untreated cells.

Treatment of adrenocortical cells by the agent may result in chronic disruption of cholesterol homeostasis that may lead to activation of the unfolded protein response (UPR) and/or triggering of the programmed cell death response and the induction apoptotic markers. The unfolded protein response is a cellular stress response activated in response to an accumulation of unfolded or misfolded proteins in the lumen of the endoplasmic reticulum. UPR halts protein translation, degrades misfolded proteins, and activates signaling pathways that lead to increased production of molecular chaperones. However, in conditions of prolonged stress, UPR initiates apoptosis. Activation of the unfolded protein response (UPR) can be demonstrated by processing of an activated XBP-1 (Xbox binding protein) splicing product that lacks a 26 nucleotide unconventional intron, causing a frame-shift. The resulting activated XBP-1 (also known as “XBP1s”) has 376 amino acids rather than the 261 amino acids of the inactive XBP-1u isoform. In certain embodiments, activation of the unfolded protein response is determined by detecting the activated XBP-1 splice variant as set forth in Example 10. An increase in UPR activity of greater than 20% is considered significant.

In certain embodiments, an agent increases UPR activity in adrenocortical cells by greater than 20%, 35%, 40%, 45%, 50%, 55%, 60%, 65%, 70%, 75%, 80%, 85%, 90%, 95%, or 100% compared to untreated adrenocortical cells.

In certain embodiments, cholesterol homeostasis may be assessed by measuring apoptosis in adrenocortical cells. Tissue sections from adrenal glands or adrenocortical cell lines may be analyzed for the presence of apoptotic markers (e.g., DNA fragmentation and/or caspase activation). Terminal deoxynucleotidyl transferase mediated dUTP nick end labeling (TUNEL) staining uses terminal deoxynucleotidyl transferase to incorporate labeled dUTP into free 3′-OH termini generated by DNA fragmentation. TUNEL staining detects late stage apoptosis. Antibodies specific for an activated form of an apoptosis-associated caspase (e.g., caspase-2, caspase-3, caspase-6, caspase-7, caspase-8, caspase-9, or caspase-10) detect early stage apoptosis. In certain embodiments, apoptosis is assessed by TUNEL staining or immunostaining for activated caspase-3 as set forth in Example 7. Apoptosis can also be assessed using a variety of commercial assay kits (e.g., ApoTox-Glo™ Triplex Assay, Promega, Madison, Wis., USA). An increase in apoptosis of greater than 20% compared to untreated cells/tissues is considered significant.

In certain embodiments, an agent increases apoptotic activity in adrenocortical cells by greater than 20%, 35%, 40%, 45%, 50%, 55%, 60%, 65%, 70%, 75%, 80%, 85%, 90%, 95%, or 100% compared to untreated adrenocortical cells.

In certain embodiments, the agent exhibits an IC₅₀ value against huACAT1 of less than 10 μM, 5 μM, 3 μM, 1 μM, 0.5 μM, 0.3 μM, 0.1 μM, 0.05 μM, 0.03 μM, 0.01 μM, 0.005 μM, 0.003 μM, or 0.001 μM. In certain embodiments, the agent exhibits an IC₅₀ value against huACAT1 from about 0.001 μM to about 10 μM, from about 0.001 μM to about 5 μM, from about 0.001 μM to about 3 μM, from about 0.001 μM to about 1 μM, from about 0.01 μM to about 10 μM, from about 0.01 μM to about 5 μM, from about 0.01 μM to about 3 μM, from about 0.01 μM to about 1 μM, from about 0.1 μM to about 10 μM, from about 0.1 μM to about 5 μM, from about 0.1 μM to about 3 μM, or from about 0.1 μM to about 1 μM.

In certain embodiments, the agent may be any one of the compounds selected from Table 1.

TABLE 1
Exemplary Compounds

| Cpd. | | |
|---|---|---|
| No. | Structure | References |
| 1 | 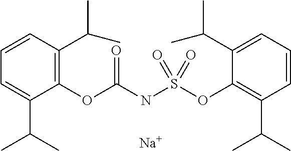 [SMILES: CC(C)c1cccc(C(C)C)c1OC(=O)NS(=O)(=O)Oc1c(C(C)C)cccc1C(C)C ; [Na+]] | Sliskovic et al., 1994, J. Med. Chem., 37:560-2; Wolfgang et al. 1995, Fundam. Appl. Toxicol. 26:272-81 |
| 2 | 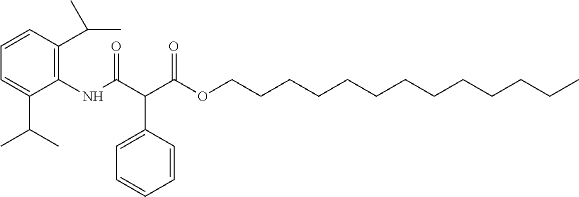 [SMILES: CCCCCCCCCCCCCOC(=O)C(C(=O)Nc1c(C(C)C)cccc1C(C)C)c1ccccc1] | Sliskovic et al., 1998, J. Med. Chem. 41:682-690 |
| 3 | 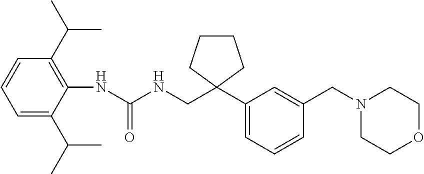 [SMILES: CC(C)c1cccc(C(C)C)c1NC(=O)NCC1(c2cccc(CN3CCOCC3)c2)CCCC1] | Trivedi et al., 1994, J. Med. Chem 37:1652-1659 |
| 4 | 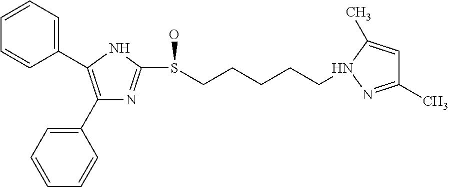 | Harris et al., 1992, J. Med. Chem 35:4384-4392; Riddell et al., 1996, Biochem. Pharmacol. 52:1177-1196; Jones et al., 1994, J. Chromatogr. B. Biomed. Appl. 661:119-31 |
| 5 | 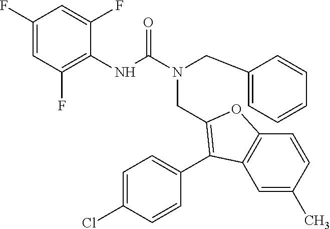 [SMILES: Cc1ccc2oc(CN(Cc3ccccc3)C(=O)Nc3c(F)cc(F)cc3F)c(-c3ccc(Cl)cc3)c2c1] | Matsuo et al., 1996, Toxicol. Pharmacol. 140:384-392 |
| 6 | 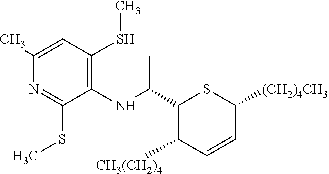 | Wilde et al., Bioorganic Med. Chem. 4:1493-1513 |
| 7 | 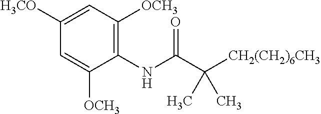 | Krause et al., 1993, J. Lipid Res. 34:279-94 |

In another aspect, the present disclosure provides a method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) reduces mitochondrial function in adrenocortical cells, and wherein the agent is not N-(2,6-bis(1-methylethyl)phenyl)-N′-((1-(4-(dimethylamino)phenyl)cyclopentyl)-methyl)urea or a salt thereof.

In this embodiment, the agent of the methods described herein inhibits the ability of huACAT1 to catalyze the esterification of free cholesterol into cholesteryl ester. An agent's inhibitory activity and IC₅₀ is measured using a fluorescent cell-based assay measuring esterification of NBD-cholesterol in AC29 cells expressing huACAT1 as described in Lada et al. (J. Lipid Res. 45:378-386, 2004) (hereby incorporated by reference in its entirety) as described in above (see also, Example 1).

The agent of the methods described herein exhibits an IC₅₀ value against human ACAT1 (huACAT1) of less than 10 μM. In certain embodiments, the agent exhibits an IC50 value against huACAT1 of less than 10 μM, 5 μM, 3 μM, 1 μM, 0.5 μM, 0.3 μM, 0.1 μM, 0.05 μM, 0.03 μM, 0.01 μM, 0.005 μM, 0.003 μM, or 0.001 μM. In certain embodiments, the agent exhibits an IC₅₀ value against huACAT1 from 0.001 μM to 10 μM, from about 0.001 μM to about 5 μM, from about 0.001 μM to about 3 μM, from about 0.001 μM to about 1 μM, from about 0.01 μM to about 10 μM, from about 0.01 μM to about 5 μM, from about 0.01 μM to about 3 μM, from about 0.01 μM to about 1 μM, from about 0.1 μM to about 10 μM, from about 0.1 μM to about 5 μM, from about 0.1 μM to about 3 μM, or from about 0.1 μM to about 1 μM.

Thus, in another embodiment, the agent of the methods described herein reduces mitochondrial function in adrenocortical cells. A reduction in mitochondrial function may be the result of mitochondrial damage or a significant decline in number. Reduction of mitochondrial function may result from disruption of mitochondrial DNA replication, inhibition of the electron transport chain, uncoupling of ATP synthesis from electron transport, inhibition of the Krebs cycle, inhibition of mitochondrial transporters, changes in mitochondrial membrane permeability, or a reduction in the number of mitochondria per cell. Mitochondrial function may be assessed by measuring cellular ATP levels. In certain embodiments, mitochondrial function is assessed using the Mitochondrial ToxGlo™ Assay (Promega, Madison Wis.) as set forth in Example 5 (Arduengo, M. Differentiating Mitochondrial Toxicity from Other Types of Mechanistic Toxicity. [Internet] May 2012. [cited: 2013, Sep. 17]. Available from: http://www.promega.com/resources/articles/pubhub/differentiating-mitochondrial-toxicity-from-other-types-of-mechanistic-toxicity/). The ToxGlo™ Assay assesses cell membrane integrity by measuring for activity of a protease released from necrotic cells, using a fluorogenic peptide substrate. The substrate cannot cross intact cell membranes of live cells and therefore only generates a signal in necrotic cells. An ATP detection reagent is added to the same cell culture well, lysing viable cells releasing ATP and generating a luminescent signal proportion to the level of ATP. The two data sets may be combined to yield an agent profile for mitochondrial toxicity or non-mitochondrial cytotoxicity. As used herein an agent that reduces mitochondrial function (e.g., decrease in ATP level) may or may not be associated with non-mitochondrial toxicity, as measured by membrane permeability. A decrease of 10% mitochondrial function in adrenocortical cells treated with an agent compared with vehicle treated controls is considered significant.

In certain embodiments, an agent reduces mitochondrial function in adrenocortical cells by greater than 10%, 15%, 20%, 25%, 30%, 35%, 40%, 45%, 50%, 55%, 60%, 65%, 70%, 75%, 80%, 85%, 90%, or 95% compared to untreated adrenocortical cells.

In certain embodiments, the agent does not induce injury or dysfunction in the liver. An agent may not induce significant effects in the liver due to preferential distribution of the agent to the adrenal gland, preferential distribution of ACAT1 in the adrenal gland, rapid clearance or metabolism of the agent in the liver, or a combination thereof. For these compounds to have their effect, they need to be at the site of action at a given threshold concentration and for a given time period to be effective. The differential effects seen between the adrenal gland and the liver can be due to the greater residence time or exposure in the adrenal vs. the liver or the presence of the target site in the adrenal and not in the liver. Morphological manifestations of liver cell effects may include cytoplasmic vacuolation, granular degeneration, necrosis, cell lysis, apoptosis, accumulation of lipid bodies, swollen endoplasmic reticulum or mitochondria, atrophy, nodular regeneration, fibrosis, hypertrophy, hyperplasia, and ultrastructural lesions of phospholipidosis. Liver cell effects are assessed by measuring apoptosis as set forth in Example 7.

In certain embodiments, the agent has low tissue concentration in the liver compared to the adrenal gland.

In certain embodiments, the agent may be any one of the compounds selected from Table 1.

In another aspect, the present disclosure provides a method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) is preferentially bound by low-density lipoprotein (LDL), and wherein the agent is not N-(2,6-bis(1-methylethyl)phenyl)-N′-((1-(4-(dimethylamino)phenyl)cyclopentyl)-methyl)urea or a salt thereof.

In this embodiment, the agent of the methods described herein inhibits the ability of huACAT1 to catalyze the esterification of free cholesterol into cholesteryl ester. An agent's inhibitory activity and IC₅₀ is measured using a fluorescent cell-based assay measuring esterification of NBD-cholesterol in AC29 cells expressing huACAT1 as described in Lada et al. (J. Lipid Res. 45:378-386, 2004) (hereby incorporated by reference in its entirety) as described above (see also, Example 1).

The agent of the methods described herein exhibits an IC₅₀ value against human ACAT1 (huACAT1) of less than 10 μM. In certain embodiments, the agent exhibits an IC₅₀ value against huACAT1 of less than 10 μM, 5 μM, 3 μM, 1 μM, 0.5 μM, 0.3 μM, 0.1 μM, 0.05 μM, 0.03 μM, 0.01 μM, 0.005 μM, 0.003 μM, or 0.001 μM. In certain embodiments, the agent exhibits an IC₅₀ value against huACAT1 from 0.001 μM to 10 μM.

Preferential binding of an agent by LDL is measured by analyzing lipoprotein fractions from a subject. Very low density lipoprotein (VLDL), high density lipoprotein (HDL), LDL, and lipoprotein free fractions are separated from a subject's plasma by ultracentrifugation using methods as described in Example 6. The amount of agent in each lipoprotein fraction is determined by LC/MS. An agent is preferentially bound by LDL if the concentration of agent in the LDL fraction exceeds the amount of agent bound by HDL and/or VLDL fractions.

An agent that preferentially binds LDL has a concentration in the LDL fraction is at least 10% greater than the concentration of the agent in the HDL or VLDL fractions. In certain embodiments, the agent has a concentration in the LDL fraction at least 20%, 30%, 40%, 50%, 60%, 70%, 80%, 90%, 100%, 150%, 200%, 250%, 300%, 350%, 400%, 450%, 500%, 600%, 700%, 800%, or 900% greater than the concentration of agent in the HDL and/or VLDL fractions. In certain embodiments, the agent has a concentration in the LDL fraction of about 10% to about 900%, of about 20% to about 900%, of about 30% to about 900%, of about 40% to about 900%, of about 50% to about 900%, of about 60% to about 900%, of about 70% to about 900%, of about 80% to about 900%, of about 90% to about 900%, of about 100% to about 900%, of about 200% to about 900%, of about 300% to about 900%, of about 10% to about 50%, of about 10% to about 100%, of about 10% to about 200%, of about 10% to about 300%, of about 10% to about 400%, of about 10% to about 500%, of about 10% to about 600%, of about 10 to about 700% greater than the concentration of the agent in the HDL and/or VLDL fractions.

In certain embodiments, the agent is preferentially distributed to the adrenal gland or the ovary, as compared to liver, kidney, skeletal muscle, and fat.

In certain embodiments, the agent may be any one of the compounds selected from Table 1.

In yet another aspect, the present disclosure provides a method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) has effects on adrenocortical cells, and wherein the agent is not N-(2,6-bis(1-methylethyl)phenyl)-N′-((1-(4-(dimethylamino)phenyl)cyclopentyl)-methyl)urea or a salt thereof.

In this embodiment, the agent of the methods described herein inhibits the ability of huACAT1 to catalyze the esterification of free cholesterol into cholesteryl ester. An agent's inhibitory activity and IC₅₀ is measured using a fluorescent cell-based assay measuring esterification of NBD-cholesterol in AC29 cells expressing huACAT1 as described in Lada et al. (2004, J. Lipid Res. 45:378-386) (hereby incorporated by reference in its entirety) as described above (see also, Example 1).

The agent of the methods described herein exhibits an IC₅₀ value against human ACAT1 (huACAT1) of less than 10 μM. In certain embodiments, the agent exhibits an IC₅₀ value against huACAT1 of less than 10 μM, 5 μM, 3 μM, 1 μM, 0.5 μM, 0.3 μM, 0.1 μM, 0.05 μM, 0.03 μM, 0.01 μM, 0.005 μM, 0.003 μM, or 0.001 μM. In certain embodiments, the agent exhibits an IC₅₀ value against huACAT1 from about 0.001 μM to about 10 μM, from about 0.001 μM to about 5 μM, from about 0.001 μM to about 3 μM, from about 0.001 μM to about 1 μM, from about 0.01 μM to about 10 μM, from about 0.01 μM to about 5 μM, from about 0.01 μM to about 3 μM, from about 0.01 μM to about 1 μM, from about 0.1 μM to about 10 μM, from about 0.1 μM to about 5 μM, from about 0.1 μM to about 3 μM, or from about 0.1 μM to about 1 μM.

Morphological markers of adrenocortical cell effects may include cytoplasmic vacuolation, granular degeneration, necrosis, cell lysis, apoptosis, accumulation of lipid bodies, swollen endoplasmic reticulum or mitochondria, atrophy, nodular regeneration, fibrosis, hypertrophy, hyperplasia, and ultrastructural lesions of phospholipidosis. In certain embodiments, adrenocortical cell effects are assessed by measuring apoptosis in adrenocortical cells. Tissue sections from adrenal glands or adrenocortical cell lines may be analyzed for the presence of apoptotic markers (e.g., DNA fragmentation and/or caspase activation). Terminal deoxynucleotidyl transferase mediated dUTP nick end labeling (TUNEL) staining uses terminal deoxynucleotidyl transferase to incorporate labeled dUTP into free 3′-OH termini generated by DNA fragmentation. TUNEL staining detects late stage apoptosis. Antibodies specific for an activated form of an apoptosis-associated caspase (e.g., caspase-2, caspase-3, caspase-6, caspase-7, caspase-8, caspase-9, or caspase-10) detect early stage apoptosis. In certain embodiments, adrenocortical cell effects are assessed by TUNEL staining or immunostaining for activated caspase-3 as set forth in Example 7. An agent has effects on adrenocortical cells if the cells exhibit an increase in a morphological marker of adrenocortical cell effects compared to vehicle treated controls. For example, an agent has effects on adrenocortical cells if the cells exhibit moderate staining or fluorescence in a TUNEL or caspase detection assay compared to vehicle treated controls. Moderate staining means that a lesion is prominent but there is significant potential for increased severity. Limited tissue or organ dysfunction is possible.

In certain embodiments, the agent may be any one of the compounds selected from Table 1.

In certain embodiments, the methods for treating a disorder associated with aberrant adrenocortical cell behavior involve administration of an agent that (a) exhibits an IC₅₀ value against huACAT1 of less than 10 μM in an assay measuring esterification of NBD-cholesterol in AC29 cells expressing huACAT1; and has one or more of the following characteristics: reduces steroid biosynthesis in adrenocortical cells; reduces mitochondrial function in adrenocortical cells as measured by cellular ATP levels; is preferentially bound by LDL as measured by LC/MS; has effects on adrenocortical cells; and disrupts cholesterol homeostasis in adrenocortical cells, wherein the agent is not N-(2,6-bis(1-methylethyl)phenyl)-N′-((1-(4-(dimethylamino)phenyl)-cyclopentyl)-methyl)urea or a salt thereof.

In certain embodiments, the present disclosure provides a method for treating a disorder associated with aberrant adrenocortical cell behavior in a subject in need thereof, comprising administering to the subject an agent that (a) exhibits an IC₅₀ value against huACAT1 of less than 10 μM in an assay measuring esterification of NBD-cholesterol in AC29 cells expressing huACAT1; and (b) and has one or more of the following characteristics: reduces steroid biosynthesis in adrenocortical cells; reduces mitochondrial function in adrenocortical cells as measured by cellular ATP levels; is preferentially bound by LDL as measured by LC/MS; has effects on adrenocortical cells; and disrupts cholesterol homeostasis in adrenocortical cells, wherein the agent is not N-(2,6-bis(1-methylethyl)phenyl)-N′-((1-(4-(dimethylamino)phenyl)-cyclopentyl)-methyl)urea or a salt thereof wherein the agent is administered concurrently or sequentially with cholesterol. In certain embodiments, the present provided herein is a method for inhibiting aberrant adrenocortical cell behavior in a subject in need thereof, comprising administering to adrenocortical cells an agent that (a) exhibits an IC₅₀ value against huACAT1 of less than 10 μM in an assay measuring esterification of NBD-cholesterol in AC29 cells expressing huACAT1; and (b) and has one or more of the following characteristics: reduces steroid biosynthesis in adrenocortical cells; reduces mitochondrial function in adrenocortical cells as measured by cellular ATP levels; is preferentially bound by LDL as measured by LC/MS; has effects on adrenocortical cells; and disrupts cholesterol homeostasis in adrenocortical cells, wherein the agent is not N-(2,6-bis(1-methylethyl)phenyl)-N′-((1-(4-(dimethylamino)phenyl)-cyclopentyl)-methyl)urea or a salt thereof; wherein the agent is administered concurrently or sequentially with cholesterol. In certain embodiments, the adrenocortical cells are H295R cells.

In certain embodiments, the concurrent or sequential administration with cholesterol increases the potency of the agent in adrenocortical cells compared to cell treated with the agent alone. In certain embodiments, the agent is administered in a sub-therapeutically effective amount. In certain embodiments, the agent may be any one of the compounds selected from Table 1.

In certain embodiments, about 5 μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, 100 μg/ml, 105 μg/ml, 110 μg/ml, 115 μg/ml, 120 μg/ml, 125 μg/ml, 130 μg/ml, 135 μg/ml, 140 μg/ml, 145 μg/ml, 150 μg/ml, 155 μg/ml, 160 μg/ml, 165 μg/ml, 170 μg/ml, 175 μg/ml, 180 μg/ml, 185 μg/ml, 190 μg/ml, 195 μg/ml, or 200 μg/ml of cholesterol is administered with the agent. In certain embodiments, about 5 μg/ml 200 μg/ml, about 10 μg/ml-100 μg/ml, or about 15 μg/ml to 60 μg/ml of cholesterol is administered with the agent.

Inhibition of ACAT1 and the subsequent disruption of cholesterol homeostasis may cause adrenocortical cells to be particularly susceptible to the addition of exogenous cholesterol. Addition of exogenous cholesterol would have the effect of increasing the potency of the agent and showing effects at concentrations below the IC₅₀ for the agent as compared to the IC₅₀ in the absence of exogenous cholesterol.

The agent and exogenous cholesterol may be administered to the adrenocortical cells or subject simultaneously in the same formulation. Alternatively, the agent and exogenous cholesterol may be administered to the adrenocortical cells or subject concurrently (within 30 minutes of each other) in separate formulations. In another aspect, the agent may be administered prior to administration of cholesterol to the adrenocortical cells or subject or subsequent to administration of cholesterol to the adrenocortical cells or subject. Prior administration of the agent refers to administration within the range of one day up to 30 minutes before administration of cholesterol to the subject. Subsequent administration of the agent refers to administration within the range of from 30 minutes up to one day after administration of cholesterol.

An increase in potency of an agent co-administered with cholesterol may be assessed by measuring a variety of endpoints including, for example, cytotoxicity (e.g., MTT assay), cell viability, membrane permeability, ATP release, activation of unfolded protein response (e.g., detection of activated XBP-1 splice variant), induction of apoptosis, etc. using commercially-available assay kits or methods known in the art. In certain embodiments, the effect of an agent combined with cholesterol is assessed as set forth in Example 11. An increase in the potency of an agent by co-administration with exogenous cholesterol of greater than 20% as compared to administration of the agent alone is considered significant. In certain embodiments, co-administration of cholesterol with an agent increases the agent's potency in adrenocortical cells by greater than 20%, 35%, 40%, 45%, 50%, 55%, 60%, 65%, 70%, 75%, 80%, 85%, 90%, 95%, or 100% compared to cells treated with the agent alone.

In certain embodiments, a disorder associated with aberrant adrenocortical cell behavior is increased hormone production, benign adenoma, adrenocortical carcinoma, metastatic adrenocortical carcinoma, congenital adrenal hyperplasia, hyperaldosteronism including Conn syndrome, a unilateral aldosterone-producing adenoma, bilateral adrenal hyperplasia (or idiopathic hyperaldosteronism (IHA)), renin-responsive adenoma, primary adrenal hyperplasia and glucocorticoid-remediable aldosteronism (GRA), 21-hydroxylase deficiency, or Cushing's syndrome.

In certain embodiments, the agent treats adrenocortical carcinoma, treats benign adenoma, treats increased hormone production, treats metastatic adrenocortical carcinoma, treats congenital adrenal hyperplasia, treats Cushing's syndrome, treats excess cortisol production, treats symptoms associated with excess cortisol production, treats hyperaldosteronism, treats 21-hydroxylase deficiency, reduces adrenocortical tumor size, or inhibits aberrant adrenal hormone production.

In some embodiments, the methods described herein further comprise administering a standard of care cancer therapy to the subject. In the context of methods of the invention, “standard of care” refers to a treatment that is generally accepted by clinicians for a certain type of patient diagnosed with a type of illness.

The compound is administered by any suitable means, either systemically or locally, including via parenteral, subcutaneous, intrapulmonary, intramuscular, oral, and intranasal. Parenteral routes include intravenous, intraarterial, epidural, and intrathecal administration. In various aspects, the compound is administered by pulse infusion. Other administration methods are contemplated, including topical, particularly transdermal, transmucosal, rectal, oral or local administration.

Another aspect of the disclosure provides a pharmaceutical composition for treating a condition.

One or more other pharmaceutically acceptable components as described in Remington's Pharmaceutical Sciences 16th edition, Osol, A. Ed. (1980) is included in the formulation provided that they do not adversely affect the desired characteristics of the formulation. Examples of formulations for a pharmaceutical composition include, without limitation, solutions, suspensions, powders, granules, tablets, capsules, pills, lozenges, chews, creams, ointments, gels, liposome preparations, nanoparticulate preparations, injectable preparations, enemas, suppositories, inhalable powders, sprayable liquids, aerosols, patches, depots and implants. In various aspects, a pharmaceutical composition formulation is in the form of a tablet or a capsule. Tablets are, in various aspects, uncoated or comprise a core that is coated, for example with a nonfunctional film or a release-modifying or enteric coating. In various aspects, capsules have hard or soft shells comprising, for example, gelatin and/or HPMC, optionally together with one or more plasticizers. Lyophilized formulations or aqueous solutions are contemplated. Sustained release formulations are also provided.

Various components of a pharmaceutical composition provided depend on the chosen route of administration and desired delivery method.

Suitable carriers include any material which, when combined with the compound, retains the activity and is nonreactive with the subject's immune system. Examples include, but are not limited to, any of a number of standard pharmaceutical carriers. A variety of aqueous carriers are contemplated and include, without limitation, water, buffered water, physiological saline, 0.4% saline, and 0.3% glycine.

In various aspects, a pharmaceutical composition formulation includes a protein for enhanced stability, such as and without limitation, albumin, lipoprotein, and globulin.

In various aspects, a pharmaceutical composition formulation includes a diluent, either individually or in combination, such as, and without limitation, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like.

Diluents, if present, typically constitute in total about 5% to about 99%, about 10% to about 85%, or about 20% to about 80%, by weight of the composition. The diluent or diluents selected exhibit suitable flow properties and, where tablets are desired, compressibility.

In various aspects, a pharmaceutical composition formulation includes binding agents or adhesives which are useful excipients, particularly where the composition is in the form of a tablet. Such binding agents and adhesives should impart sufficient cohesion to the blend being formulated in a tablet to allow for normal processing operations such as sizing, lubrication, compression and packaging, but still allow the tablet to disintegrate and the compound to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC or hypromellose), hydroxypropyl-cellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, constitute in various aspects, in total about 0.5% to about 25%, for example about 0.75% to about 15%, or about 1% to about 10%, by weight of the composition.

In various aspects, an aqueous pharmaceutical composition formulation of the compound includes a buffer. Examples of buffers include acetate (e.g., sodium acetate), succinate (such as sodium succinate), gluconate, histidine, citrate and other organic acid buffers. The buffer concentration can be from about 1 mM to about 200 mM, or from about 10 mM to about 60 mM, depending, for example, on the buffer and the desired isotonicity of the formulation. In various aspects, an aqueous pharmaceutical composition formulation of the compound is prepared in a pH-buffered solution, for example, at pH ranging from about 4.5 to about 8.0, or from about 4.8 to about 6.5, or from about 4.8 to about 5.5, or alternatively about 5.0.

In various aspects, a pharmaceutical composition formulation includes a disintegrant.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

In various aspects, a pharmaceutical composition formulation includes a wetting agent. Wetting agents, if present, are normally selected to maintain the compound in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

In various aspects, a pharmaceutical composition formulation includes a lubricant. Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

In various aspects, a pharmaceutical composition formulation includes an anti-adherent. Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

In various aspects, a pharmaceutical composition formulation includes a glidant. Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

In various aspects, a pharmaceutical composition formulation includes a tonicity agent. A tonicity agent may be included in the formulation for stabilization. Exemplary tonicity agents include polyols, such as mannitol, sucrose or trehalose. Preferably, the aqueous formulation is isotonic, although hypertonic or hypotonic solutions are contemplated. Exemplary concentrations of the polyol in the formulation may range from about 1% to about 15% w/v.

In various aspects, a pharmaceutical composition formulation includes a surfactant. A surfactant may also be added to reduce aggregation of the compound and/or to minimize the formation of particulates in the formulation and/or to reduce adsorption. Exemplary surfactants include nonionic surfactants such as polysorbates (e.g., polysorbate 20 or polysorbate 80) or poloxamers (e.g., poloxamer 188). Exemplary concentrations of surfactant may range from about 0.001% to about 0.5%, or from about 0.005% to about 0.2%, or alternatively from about 0.004% to about 0.01% w/v.

In various aspects, a pharmaceutical composition formulation is essentially free of one or more preservatives, such as benzyl alcohol, phenol, m-cresol, chlorobutanol and benzethonium. In other aspects, a preservative is included in the formulation, e.g., at concentrations ranging from about 0.1% to about 2%, or alternatively from about 0.5% to about 1%.

Sustained-release pharmaceutical composition formulations are also provided. Suitable examples of sustained-release preparations include semipermeable matrices of solid hydrophobic polymers containing the antibody, which matrices are in the form of shaped articles, including without limitation films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The active ingredients may also be entrapped in a microcapsule prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Also provided are pharmaceutical compositions in a lyophilized formulation. The resulting “lyophilized cake” is reconstituted prior to use. Reconstitution of the lyophilized cake adds a volume of aqueous solution, typically equivalent to the volume removed during lyophilization.

The amount of the compound to be administered, and other administration parameters such as frequency and duration of therapy, depend on the compound or prodrug intended for use, and on other factors such as the route of administration, dose intervals, excretion rate, formulation of the compound, the recipient, age, body weight, sex, diet, medical history, and general state (e.g., health) of the subject being treated of the recipient, the severity of the disease, and/or the size, malignancy and invasiveness of a tumor to be treated The compound is thus administered at a dosage sufficient to achieve a desired therapeutic or prophylactic effect and is determined on a case-by-case basis.

In some embodiments, the compound is administered at a dosage of about 1.0 μg/kg to about 100 mg/kg, about 0.01 mg/kg to about 100 mg/kg, about 0.01 mg/kg to about 50 mg/kg, about 0.01 mg/kg to about 30 mg/kg, about 0.01 mg/kg to about 10 mg/kg, about 0.01 mg/kg to about 5 mg/kg, about 0.05 mg/kg to about 10 mg/kg, about 0.1 mg/kg to about 200 mg/kg, about 0.1 mg/kg to about 50 mg/kg, about 0.1 mg/kg to about 10 mg/kg, about 0.5 mg/kg to about 25 mg/kg, about 1 mg/kg to about 10 mg/kg, or about 2 mg/kg to about 10 mg/kg.

Administration is contemplated in a regiment that is daily (once, twice or more per day), alternating days, every third day, or 2, 3, 4, 5, or 6 times per week, weekly, twice a month, monthly or more or less frequently, as necessary, depending on the response or condition and the recipient tolerance of the therapy. Maintenance dosages over a longer period of time, such as 4, 5, 6, 7, 8, 10 or 12 weeks or longer are contemplated, and dosages may be adjusted as necessary. The progress of the therapy is monitored by conventional techniques and assays, and is within the skill in the art.

The following examples are given merely to illustrate the present invention and not in any way to limit its scope.

### EXAMPLES

### Example 1

### ACAT 1 Inhibition

Human ACAT1 activity was measured as described by Lada et al., 2004, J. Lipid Res. 45:378-386. AC29 cells (a Chinese hamster ovary cell-derived cell line) lack any endogenous ACAT1 activity and were used as the recipient cell line for these experiments (Cadigan et al., 1988, J. Biol. Chem. 263:274-282). Stable transfectants expressing recombinant human ACAT1 or ACAT2 genes encoding a polypeptide sequence of Genbank Accession # AAC37532.2 (SEQ ID NO:2) or Genbank Accession #AAC63998.1 (SEQ ID NO:3), respectively were created as described in Lada et al. (supra). Cells were maintained in monolayer at 37° C. in 5% CO₂ in Ham's F-12 medium supplemented with 1% Eagle's vitamins, penicillin (100 units/ml), streptomycin (100 μg/ml), and 10% heat-inactivated fetal bovine serum, and cells were typically grown to 70-90% confluence for all experiments.

Human ACAT1 activity was assessed using a fluorescent cell-based assay as described in Lada et al. (supra). The assay uses 22-[N-(7-nitrobenz-2-oxa-1,3-diazol-4-yl)amino]-23,24-bisnor-5-cholen-3-ol (NBD-cholesterol), a fluorescent sterol analog in which the NBD moiety replaces the terminal segment of the alkyl tail of cholesterol. NBD-cholesterol has been shown to mimic native cholesterol absorption in multiple systems. In a polar environment, NBD-cholesterol is weakly fluorescent. In a nonpolar environment, NBD-cholesterol strongly is strongly fluorescent. The fluorescent property of NBD-cholesterol is used to measure ACAT activity, as cholesterol is a polar lipid and cholesteryl ester is nonpolar. 3×10⁵ cells per well were plated on Falcon 96-well culture plates and allowed to recover overnight. Assays were performed with cells that are at least 80% confluent. Media was aspirated and wells were pre-incubated with 100 μL of dilutions of control and test compounds from 1000× stock solutions in DMSO (or other neutral diluent e.g., methanol, ethanol, or water) for 20 minutes. Media was removed and replaced with 100 μL of medium containing the same dilution of compound or vehicle as in the pre-incubation and 1 μg/ml NBD-cholesterol and incubated for 6 hr at 37° C. (Lada et al., supra). NBD-cholesterol was added from a 1 mg/ml stock in ethanol (final ethanol concentrations did not exceed 0.1%). Parental AC29 cells incubated with NBD-cholesterol or ACAT1-expressing cells incubated with NBD-cholesterol and the vehicle or control compounds were used to determine background fluorescence attributable to free NBD-cholesterol. After incubation, medium is removed, and the cells were washed two times with cold balanced salt solution (BSS). Plates were read from the bottom using a Tecan GENios fluorescent plate reader equipped with 485 nm excitation and 535 nm emission filters. After measurement, cellular protein was digested through incubation with 25 μl of 0.4 N NaOH for 2 h. Protein content was determined by the method of Lowry et al. (1951, J. Biol. Chem. 193:265-275). Cholesteryl ester (CE) fluorescence was calculated by subtracting the background from the total fluorescence, and these values were normalized by cellular protein. Data were plotted using an appropriate graphing program (e.g., GraphPad Prism®) to calculate the IC₅₀ (concentration at 50% inhibition) for each test compound (see Table 2).

TABLE 2
ACAT IC₅₀ Values for Compounds Tested

| | IC₅₀ huACAT1 | IC₅₀ huACAT1 |
|---|---|---|
| Cpd. No. | (μM) | (μM) |
| 1 | 2.4 | 0.44 |
| | n = 1 | n = 1 |
| 2 | 0.02 | 0.021 |
| | n = 1 | n = 1 |
| 3 | 2.0 | 0.51 |
| | n = 2 | n = 2 |
| 4 | 0.45 | 0.24 |
| | n = 1 | n = 1 |
| 5 | 0.006 | 0.08 |
| | n = 1 | n = 1 |
| 6 | 0.0018 | 0.018 |
| | n = 1 | n = 1 |
| 7 | 0.54 | NC* |
| | n = 1 | n = 1 |

*NC = not calculated

### Example 2

### Reduction of Steroid Biosynthesis

The test agent formulation(s) and appropriate vehicle controls are administered by oral (gavage) to beagle dogs for 14 consecutive days. The dose level, formulation, volume, route, and frequency of administration are appropriate for the test compound. Blood is collected from all animals on Day −3, and all surviving animals on Days 1, 3, 7, 8, 10, and 14. 5 micrograms/kg (not to exceed 250 micrograms) of CORTROSYN™ (also known as cosyntropin or synthetic ACTH) are administered via bolus IV at approximately the same time each morning. Blood samples are collected pre-dose and 1 hour post-ACTH-dose at approximately the same time each morning (±1 hour from the Day 1 collection times), including Day −3. After coagulation, serum is harvested and frozen (−50 to −90° C.) until analysis.

Samples are analyzed by ELISA using Canine Cortisol Quantitative ELISA Kit (Endocrine Technologies, Cat. # ERK-C2003) for the measurement of cortisol in canine serum and plasma. Kit components are stored and expire as assigned by the manufacturer. Antibody coated plates are provided ready to use in the kit. Standard/Calibrators, sample diluent, enzyme-antibody conjugate, 20× wash buffer concentrate, TMB substrate, 2N HCl stop solution, are provided ready to use in kit. Reagents are warmed to room temperature prior to use. The lowest standard provided in the kit is further diluted with Sample Diluent to obtain an additional standard point with a concentration of approximately 0.5 ng/mL. Canine serum or plasma samples are generally tested undiluted. If results are found to be above the quantifiable range of the assay (ALQ), samples may be diluted with Sample Diluent to bring the results within the range of the assay. 25 μL of the prepared standards in triplicate wells and samples in duplicate wells are added to the antibody coated plate. 25 μL of the Sample Diluent in triplicate wells are added to the plate as a blank and 100 μL of Enzyme-Antibody Conjugate are added to each well. The plates are sealed and incubated at room temperature for 60 minutes. The plates are washed with 1× Wash Buffer and the plates are patted on absorbent material to remove residual liquids. 100 μL of TMB Substrate are added to each well. The plates are sealed and incubated at room temperature for 20 minutes. 50 μl of Stop Solution are added to each well and gently mixed until the color is uniform. The plate absorbance is read at 450 nm using a spectrophotometer. Sample concentration is calculated using a standard curve constructed from the standards' concentrations and the absorbance values. A decrease in cortisol levels of greater that 10% compared to vehicle-treated controls is considered significant.

Samples are also analyzed for steroid intermediate and end products including cortisol, cortisone, corticosterone, 11-deoxycortisol, androstenedione, testosterone, 17-OH-progesterone, 11-deoxycorticosterone, progesterone, and DHEA-S by liquid chromatography/mass spectrometry (LC/MS). A 10-4 aliquot of serum is diluted with 90 μL of water in a 1.5-mL centrifuge tube. Protein is precipitated by the sequential addition of 0.2 mL acetonitrile and 0.1 mL methanol. After the addition of 0.1 mL of 1000 pg/mL internal standard (e.g., [²H₄]cortisol (C/D/N Isotopes)) in acetonitrile, the suspension is vortex mixed and centrifuged for 5 minutes at 13,000 rpm. The supernatant is transferred to a 2-mL tube with 0.5 mL of water, and steroids are extracted with 1 mL of methyl-t-butyl ether. The organic phase is transferred to a clean 2-mL tube and concentrated under nitrogen, and the dried extract is reconstituted with 0.1 mL of 50% aqueous methanol and transferred to a 0.25-mL vial insert. Samples are analyzed with an Agilent 1290 HPLC and 6490 triple quadrupole liquid chromatography-tandem mass spectrometer (Agilent Technologies) using electrospray ionization in positive ionization mode. Steroids are resolved on a Kinetex 50×2.1 mm, 2.6-μm particle size C8 column (Phenomenex) using gradient elution with 10 mM ammonium acetate and methanol. Quantitation is achieved by external standardization with a 9-point calibration curve using multiple reaction monitoring mode. Dose-dependent changes of at least 10% in one or more of these steroid intermediates or end products either an increase in an upstream substrate or a decrease in a downstream product as a result of a block at a specific step in steroid biosynthesis—as compared to vehicle control are considered to be significant.

### Example 3

### Reduction of Cortisol Biosynthesis

The test agent formulation(s) and appropriate vehicle controls are administered by oral (gavage) to beagle dogs for 14 consecutive days. The dose level, formulation, and volume and route of administration are appropriate for the test compound. Blood is collected from all animals on Day −3, and all surviving animals on Days 1, 3, 7, 8, 10, and 14. Samples are collected at approximately the same time each morning (±1 hour from the Day 1 collection times), including Day −3. After coagulation, serum is harvested and frozen (−50 to −90° C.) until analysis.

ACTH levels are determined by radioimmunoassay. The radioimmunoassay (RIA) for the detection of ACTH in canine plasma is based on a ligand-binding assay and is provided as a kit from MP Biomedicals, LLC (Catalog #07106102). Reagents, antibodies, controls, ¹²⁵I-ACTH are reconstituted according to kit instructions and stored appropriately. All tubes, samples, and reagents are maintained on ice throughout the assay unless indicated otherwise in the kit instructions. Samples are generally tested undiluted, but if samples result or are expected to result above the range of the assay, samples may be diluted. 100 μL of standards and controls are added to uncoated reaction tubes in triplicate and samples in duplicate. 100 μL of Anti-ACTH are added to all standard, control and sample tubes. 100 μL of ¹²⁵I-ACTH are added to all tubes and mixed. 100 μL of ¹²⁵I-ACTH are added only to 2 uncoated reaction tubes for the Total Count (T) tubes. Tubes are covered and incubated at 2-8° C. for 16-24 hours. 500 μL of Precipitant Solution are added to all tubes with the exception of the Total Count tubes and mixed until the color is uniform throughout the tube. All tubes are centrifuged with the exception of the Total Count tubes at 950-1050×g, 2-8° C., for 10-15 minutes. Supernatant is aspirated from all tubes, with the exception of the Total Count tubes (taking care not to disturb the pellet), and the CPM (Counts per Minute) of all tubes is determined on a gamma counter for 2 minutes. The sample concentration is calculated using a standard curve constructed from the standards' concentrations and the counts per minute (CPM) values. Increases in ACTH levels of greater than 10% compared to vehicle-treated animals are considered to be significant.

A reduction in cortisol level may also be assessed in vitro. H295R adrenocortical carcinoma (ACC) cells are obtained from the American Type Culture Collection (ATCC® CRL-2128™). Cells are maintained in growth media—DME/F12 Medium with HEPES (Gibco #11330-057) supplemented with 1% Antibiotic Solution (Pen/Strep #15070-63), 5% NuSerum I (Collaborative Research #55000), 0.00625 mg/ml insulin, 0.00625 mg/ml transferring, and 6.25 ng/ml selenium. For experiments, cells are trypsinized (0.25% Trypsin/EDTA—Gibco), centrifuged, re-suspended in growth media, and counted using a hemocytometer.

400,000 cells are added to each well of a 12-well culture plate and allowed to adhere. Stock solutions of control and test compounds are prepared to allow testing at final concentrations up to 30 μM. Dilutions of the control and test compounds are prepared separately. Aliquots of stock control and test compounds are added to individual wells of the plate to achieve final concentrations ranging from 0.01-30 μM. Reagents are mixed by placing the plate on an orbital shaker at 500-700 rpm for 1 minute. The plates are incubated at 37° C. in a humidified and CO₂-supplemented incubator for 15 minutes to 48 hours. Cortisol concentrations in the medium are determined using an ELISA (Alpco Diagnostics, Salem, N.H., USA Catalog #11-CORHU-E01) following the manufacturer's recommendations, except that standard curves are prepared in the experimental cell culture medium. Results are plotted as a percent of the vehicle control. A decrease in cortisol levels of 10% for test compounds compared to vehicle is considered significant.

### Example 4

### Reduction of Cholesterol Esters

The test agent formulation(s) and appropriate vehicle controls are administered by oral (gavage) to beagle dogs for 14 consecutive days. The dose level, formulation, and volume and route of administration are appropriate for the test compound. After treatment, all animals are euthanized approximately 4 hours post dose on Day 14 and subjected to a necropsy examination and collection of tissues. Adrenal glands are harvested, weighed, flash frozen in liquid nitrogen, and stored at −80° C. until analyzed.

Total cholesterol, free cholesterol, and by subtraction cholesteryl ester, are determined according to the method of Carr et al. (1993, Clin. Biochem. 26:39-42). Approximately 200 mg of tissue (wet weight) is weighed and pulverized in liquid nitrogen. Lipids are extracted in 30 mL chloroform:methanol (2:1) according to the method of Folch et al. (1957, J. Biol. Chem. 224:497-509). Phases are separated by the addition of 6 mL of 0.05% H₂SO₄ and the lower phase volume was recorded.

An appropriate amount of sample, estimated to contain 10-200 μg of cholesterol is pipetted into a 16×100 mm screw cap tube. One mL of Triton X-100 solution (1% v/v in chloroform) is pipetted into each tube. The solvent is dried under nitrogen at 45° C. and the sides of the tubes are washed with chloroform to concentrate the samples at the bottom of the tubes. The tubes are allowed to cool to room temperature and 0.5 mL of deionized water is added to each tube. The tubes are capped and placed in a shaking water bath at 37° C. for 15 minutes to solubilize the material. The tubes are removed from the water bath, vortexed, and enzymatic determination of total cholesterol and free cholesterol is carried out.

Total cholesterol is determined using Cholesterol High-Performance ‘Single Vial’ reagent (Boehringer Mannheim Diagnostics, Indianapolis, Ind., USA, Cat. No. 236691) according to the manufacturer's instructions. Free cholesterol is measured using Free Cholesterol C enzymatic reagent (Wako Pure Chemical Industries, Ltd., Osaka, Japan, Cat. No. 274-47109) according to the manufacturer's instructions. Cholesteryl ester (μg/mg protein) is determined by: Total cholesterol μg/mg protein minus Free cholesterol μg/mg protein. A 10% decrease in the amount of cholesteryl ester in test compound-treated adrenal glands compared to vehicle-treated control adrenal glands is considered significant.

### Example 5

### Reduction of Mitochondrial Function

H295R adrenocortical carcinoma (ACC) cells are obtained from the American Type Culture Collection (ATCC® CRL-2128™). Cells are maintained in growth media—DME/F12 Medium with HEPES (Gibco #11330-057) supplemented with 1% Antibiotic Solution (Pen/Strep #15070-63), 5% NuSerum I (Collaborative Research #55000) and 0.00625 mg/ml insulin; 0.00625 mg/ml transferrin; 6.25 ng/ml selenium. For experiments, cells are trypsinized (0.25% Trypsin/EDTA—Gibco), centrifuged, re-suspended in growth media, and counted using a hemocytometer.

50 μl of cells at 200,000 cells/ml, are added to each well of a 96-well culture plate and allowed to adhere. Cells may be cultured in high glucose, low glucose, or galactose containing media in order to improve detection of drugs that affect mitochondrial function. Under high glucose conditions, cell lines may have high glycolytic activity, rendering them more resistant to mitochondrial toxins. Stock solutions of control and test compounds are prepared to allow testing at final concentrations of 30 mM. Dilutions of the control and test compounds are prepared in a separate 96-well plate. 50 μl of diluted compounds are transferred from the stock plate to cells in culture. Reagents are mixed by placing the plate on an orbital shaker at 500-700 rpm for 1 minute. The plates are incubated at 37° C. in a humidified and CO₂-supplemented incubator for 15 minutes to 24 hours.

Mitochondrial function is measured using Mitochondrial ToxGlo™ Assay (Promega Part# G8000). Briefly, the Cytotoxicity and ATP Detection Reagents are prepared according to the manufacturer's instructions. At the end of the incubation, 20 μl of 5× Cytotoxicity Reagent is added to each well. The plate is mixed briefly (1 minute) by orbital shaking (500-700 rpm) followed by incubation at 37° C. for 30 minutes. The assay plate is equilibrated to room temperature for 5-10 minutes after which 100 μl of ATP Detection Reagent is added to each well and mixed by orbital shaking (500-700 rpm) for 1-5 minutes. The plate is read for fluorescence at 485 nmEx/520-530 nmEm. The signal should be 60-90% of detector saturation for optimal results. The plate is then read for luminescence at 60-90% of detector saturation for optimal results. Results are plotted as a percent of the vehicle control. An decrease in mitochondrial function of at least 10% for test compounds compared to vehicle is considered significant.

### Example 6

### Binding to LDL

The test agent formulation(s) and appropriate vehicle controls are administered by oral (gavage) to beagle dogs for 14 consecutive days. The dose level, formulation, volume, route and frequency of administration are appropriate for the test compound. Blood is collected from all animals on Day −3, and all surviving animals on Days 1, 3, 7, 8, 10, and 14. Plasma is harvested and stored at −80° C. until analyzed.

Lipoproteins are separated from plasma by sequential ultracentrifugation using a CS120FX ultracentrifuge and S100AT5 rotor (Hitachi Koki Co., Ltd., Tokyo, Japan). Density is adjusted with a solution (195 mM NaCl, 0.01% EDTA) containing NaBr as follows: d<1.006, VLDL (very low density lipoprotein); 1.006<d<1.063, LDL (low density lipoprotein); 1.063<d<1.21, HDL (high density lipoprotein); d>1.21, lipoprotein-free fraction. One mL of NaBr solution (d=1.006) is layered over 2 ml of plasma and centrifuged at 16° C. for 4 hr at 100,000 rpm (550,000×g). An 0.8-ml amount of the floated fraction is collected (VLDL) and after thoroughly re-suspending the pelleted lipoproteins at the bottom of the tube, the density of the remaining solution (2 ml) is adjusted with the NaBr solution (1 ml) to 1.063. LDL fraction is obtained after centrifugation as above and the density of the remaining fraction is adjusted to 1.21. After centrifugation for 16 hr, HDL and lipoprotein-free fractions are obtained as the floated (0.8 ml) and the remaining fractions, respectively.

The amount of test compound in each fraction is determined by an appropriate liquid chromatography-mass spectrometry (LC-MS) method developed and validated for each test compound. Results are calculated using peak area ratios of analytes to internal standard, and calibration curves were generated using a weighted (1/x2, where x=concentration) linear least-squares regression.

### Example 7

### Adrenocortical Cell Effects

The test agent formulation(s) and appropriate vehicle controls are administered daily by oral (gavage) to beagle dogs for 14 consecutive days. The dose level, formulation, and volume and route of administration are appropriate for the test compound. After treatment, all animals are euthanized approximately 4 hours post dose on Day 14 and subjected to a necropsy examination and collection of tissues. Adrenal glands are harvested, weighed, and placed in a neutral formalin solution in preparation for histological examination.

Tissues samples are embedded in paraffin and sections are stained using antibodies to TUNEL and Caspase-3/7 as markers of apoptosis. Sections are read and increased (e.g., moderate) staining of adrenocortical cells in test compound-treated animals compared to vehicle control-treated animals is indicative of induction of apoptosis.

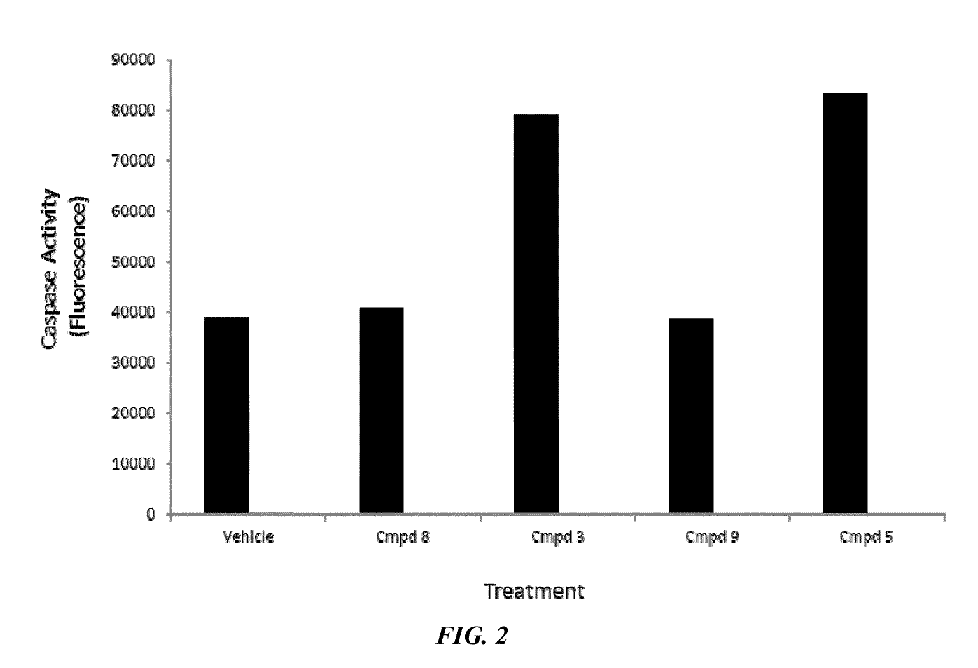

Commercially-available assay kits may also be employed to measure Caspase 3/7 activity, including for example the Apotox-Glo Assay Kit (Promega). FIG. 2 shows the effects of adrenal acting and non-adrenal acting ACAT1 inhibitors on Caspase-3/7 activity, as a measure of apoptosis, in H295R cells. H295R cells are plated at 500,000 cells/well in a 12 well plate. After overnight incubation at 37° C., media is removed and replaced with media containing vehicle (DMSO) or one of the ACAT1 inhibitors at its ACAT1 IC₅₀ concentration in the presence of 45 ug/mL exogenous cholesterol. After 5 hours of treatment, cells are harvested, washed and assayed for Caspase-3/7 activity using the Apotox-Glo Assay Kit (Promega). Both of the ACAT1 adrenal acting compounds (Compound Nos. 3 and 5 see Example 9 below), showed significant increases in Caspase-3/7 activity indicating increased apoptosis in these cells compared to vehicle. Neither of the ACAT1 non-adrenal acting compounds (Compound Nos. 8 and 9 see Example 9 below) showed increased Caspase-3/7 activity compared to vehicle indicating that apoptotic programmed cell death pathway has not been activated. Although showing similar activity on ACAT1 in in vitro assays, this result shows a differentiation of adrenal acting and non-adrenal acting compounds in this in vitro endpoint that may explain the different adrenal activities observed in vivo.

### Example 8

### Effects on Cholesterol Homeostasis

H295R adrenocortical carcinoma (ACC) cells (ATCC® CRL2128™) are maintained in growth media as described in Example 5 and plated at 500,000 cells/well in a 12-well culture plate. After overnight incubation at 37° C., media is removed and replaced with treatment media containing the agent or control in varying dilutions for a dose response. After treatment of cells for various lengths of time (minutes to hours, e.g., 15 minutes to 24 hours), RNA is extracted using the Qiagen RNEasy® kit and 1 μg RNA is reverse transcribed using Applied Biosystems® High-Capacity cDNA Reverse Transcription Kit. An agent's effect on cholesterol homeostasis is determined by measuring expression changes in calcium responsive target genes, which are amplified by PCR using gene-specific primers for CYP11B2, MC2R, NR4A2 (Nurr1), MRAP, and INHBA under the following conditions: 1) 95° C. for 30 sec; 2) 95° C. for 15 sec; 3) 50° C. for 15 sec; and 4) 68° C. for 30 sec. Amplification steps 2 through 4 are repeated for 30 cycles. PCR products are resolved on a 5% agarose gel. Changes in at least 2-fold up or down is considered to be significant.

### Example 9

### Effect of Representative Adrenal Acting and Non-Adrenal Acting ACAT1 Inhibitors on Cholesterol Homeostasis

To study the effect of adrenal acting and non-adrenal acting ACAT 1 inhibitors on cholesterol homeostasis, the levels of free and esterified cholesterol were assessed in a human adrenocortical cell line (H295R) after treatment with compound and in the context of additional exogenous cholesterol.

In this study, H295R were plated at 200,000 cells/well in a 24 well plate. After overnight incubation at 37° C., media was removed and replaced with treatment media (test compound at its ACAT1 IC₅₀ concentrations containing 45 μg/ml cholesterol) or vehicle (DMSO). After treatment of cells for various lengths of time (minutes to hours), media was removed and cells are washed twice with cold PBS. 200 ul of hexane/isopropanol (3:2) was then added to each well, the plate was rocked at room temperature for 1 hour, and the supernatant was transferred to glass vials and allowed to air dry. Residual lipids were resuspended and total cholesterol and free cholesterol were measured using the Cholesterol Fluorometric Assay Kit (Cayman Chemical). Cholesterol ester was determined by subtracting free cholesterol from total cholesterol.

TABLE 3
ACAT1 inhibitors used in H295R activity assays

| Cpd. | ACAT1 IC₅₀ | | Adrenal |
|---|---|---|---|
| No. | (μM)¹ | Structure | activity |
| 3 | 0.011 | 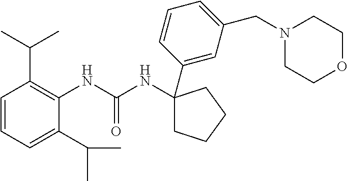 [SMILES: CC(C)c1cccc(C(C)C)c1NC(=O)NC1(c2cccc(CN3CCOCC3)c2)CCCC1] | Adrenal acting |
| 5 | 0.010 | 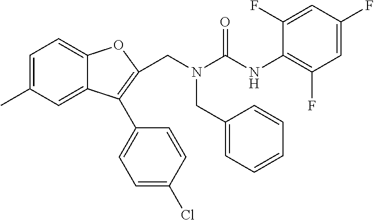 [SMILES: Cc1ccc2oc(CN(Cc3ccccc3)C(=O)Nc3c(F)cc(F)cc3F)c(-c3ccc(Cl)cc3)c2c1] | Adrenal acting |
| 8 | 0.010 | 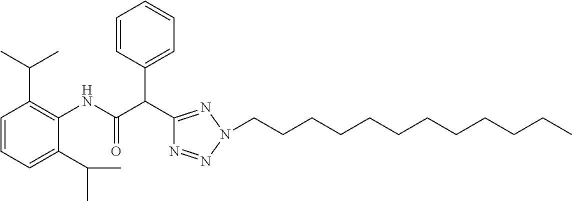 [SMILES: CCCCCCCCCCCCn1nnc(C(C(=O)Nc2c(C(C)C)cccc2C(C)C)c2ccccc2)n1] | Non-adrenal acting |
| 9 | 0.063 | 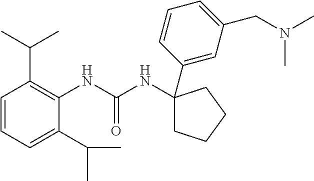 [SMILES: CC(C)c1cccc(C(C)C)c1NC(=O)NC1(c2cccc(CN(C)C)c2)CCCC1] | Non-adrenal acting |

¹IC₅₀ determinations performed as described in Example 1.
Cpd. No. 3: CAS No. 167108-30-7; 2H-Tetrazole-5-acetamide, N-[2,6-bis(1-methylethyl)phenyl]-2-dodecyl-a-phenyl-.
Cpd. No. 5: CAS No. 133825-84-0; N-[2,6-bis(1-methylethyl)phenyl]-N′-[[1-[3-[(dimethylamino)methyl]phenyl]cyclopentyl]methyl]-.
Cpd. No. 8: CAS No. 156086-41-8; Urea, N-[2,6-bis(1-methylethyl)phenyl]-N′-[[1-[3-(4-morpholinylmethyl)phenyl]cyclopentyl]methyl]-.
Cpd. No. 9: CAS No. 146011-65-6; Urea, N-[[3-(4-chlorophenyl)-5-methyl-2-benzofuranyl]methyl]-N-(phenylmethyl)-N′-(2,4,6-trifluorophenyl)-.

The compounds assessed in this study are shown in Table 3 above. As reported in the literature, and as noted in Table 3, Compound Nos. 3 and 5 are both known to be adrenal acting ACAT1 inhibitors, while Compound Nos. 8 and 9 are known to be non-adrenal acting ACAT1 inhibitors (see, e g., Sliskovic D. et al., Prog. Med. Chem. 39:121-171, 2002; Dominick M A, et al., Fund Appl Toxicol. 20:217-24, 1993; Trivedi B K, et al., J. Med. Chem. 37:1652-1659, 1994; Matsuo M, et al., Toxicol. Appl. Pharmacol. 140:387-392, 1996)(each of which is hereby incorporated by reference in its entirety).

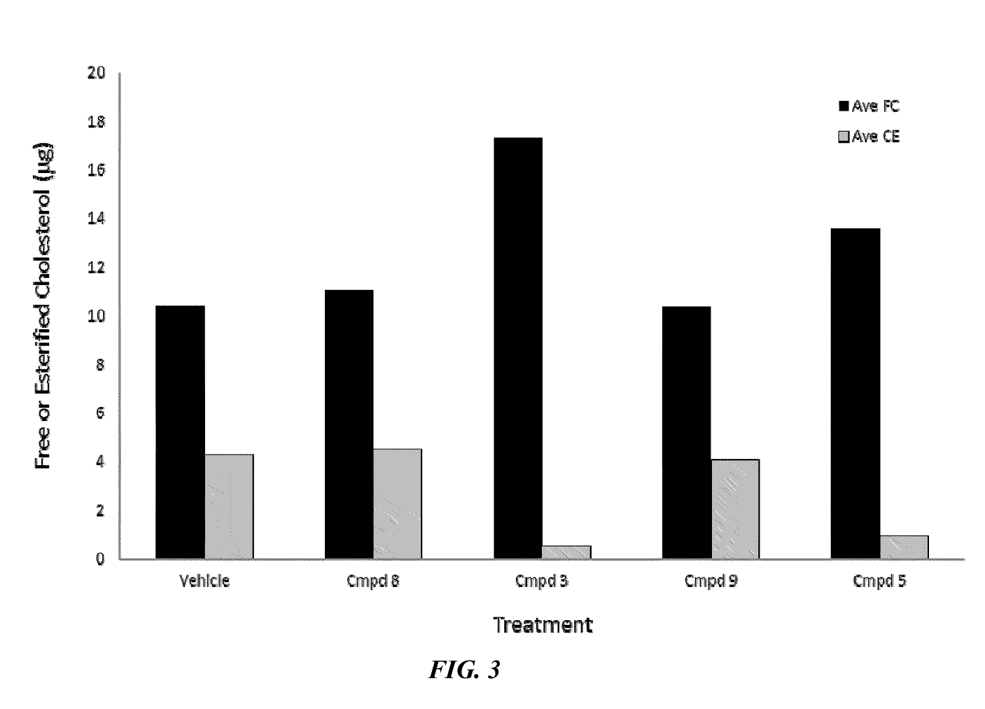

Referring to FIG. 3, both of the adrenal acting compounds showed a significant decrease in esterified cholesterol (gray bars) and an increase in free cholesterol (black bars), whereas the non-adrenal acting compounds showed no or little change in both free and esterified cholesterol compared to vehicle. Such results were observed even though all four of these compounds are potent inhibitors of ACAT1 in an in vitro assay. The increase in free cholesterol in H295R cells treated with Compound Nos. 3 and 5 give rise to other physiological changes in the cell resulting in apoptosis and adrenal acting activity observed in vivo.

### Example 10

### Effects on Unfolded Protein Response

Treatment of H295R cells by an agent can result in chronic disruption of cholesterol homeostasis that may lead to activation of the UPR and/or triggering of the programmed cell death response and the induction apoptotic markers. Activation of the unfolded protein response (UPR) can be demonstrated by induction of the XBP-1 spicing product.

H295R adrenocortical cells are plated at 500,000 cells/well in a 12-well culture plate as described in Example 8. After overnight incubation at 37° C., media is removed and replaced with media containing vehicle or an ACAT 1 inhibitor at its ACAT1 IC₅₀ concentration in the presence of 45 ug/mL exogenous cholesterol. After 5 hours of treatment, RNA is extracted using the Qiagen RNEasy® Plus kit and 1 μg RNA is reverse transcribed using Applied Biosystems® High-Capacity cDNA Reverse Transcription Kit. XBP-1 splicing product is amplified by PCR using primer pairs that allow detection of the activated isoform (5′-TTACGAGAGAAAACTCATGGCC-3′ (SEQ ID NO:15) and 5′-GGGTCCAAGTTGTCCAGAATGC-3′ (SEQ ID NO:16)) under the following conditions: 1) 95° C. for 30 sec, 2) 95° C. for 15 sec; 3) 50° C. for 15 sec, 4) 68° C. for 30 sec. Examples of XBP-1 primers specific for the inactive, unspliced XBP-1 and activated, spliced XBP-1 isoforms are described in Majumder et al. (2012, Mol. Cell. Biol. 32:992-1003). Amplification steps 2 through 4 are repeated for 30 cycles. PCR product is resolved on a 5% agarose gel. DTT treated cells serve as a positive control for XBP-1 splicing. An increase in the XBP-1 splice variant expression of greater than 20% is considered significant.

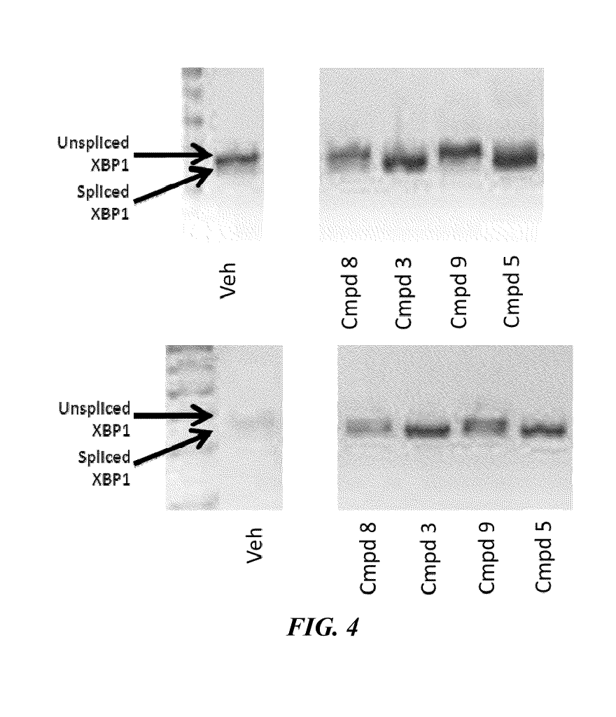

FIG. 4 shows the effects of adrenal acting and non-adrenal acting ACAT1 inhibitors on XBP-1 splicing in H295R cells in the presence of exogenous cholesterol in two separate experiments. Both of the representative ACAT1 adrenal acting compounds (Compounds 3 and 5) show significant splicing of XBP-1 indicative of activation of the UPR which could lead to downstream apoptosis. Neither of the representative ACAT1 non-adrenal acting compounds (Compounds 8 and 9) showed significant splicing of XBP-1 compared to vehicle, indicating UPR has not been activated. Although showing similar activity on ACAT1 in in vitro assays, this result shows a differentiation of adrenal acting and non-adrenal acting compounds in this in vitro endpoint consistent with the different adrenal activities observed in vivo.

### Example 11

### Effects of Cholesterol on Potency of Agent

H295R adrenocortical cells are plated at 500,000 cells/well in a 12-well culture plate as described in Example 8. After overnight incubation at 37° C., media is removed and replaced with media containing the agent at various concentrations and cholesterol at various concentrations. After varying treatment times, the effects of combination treatment with the agent and cholesterol are measured using commercially-available assay kits (e.g., Vybrant® MTT Cell Proliferation Assay Kit (Life Technologies) or CellTiter 96® Non-Radioactive Cell Proliferation Assay (Promega Corp.)). An increase in cytotoxicity as measured by the MTT assay of greater than 20% is considered significant.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) has one or more effects on adrenocortical cells, and wherein the agent is not N-(2,6-bis(1-methylethyl)phenyl)-N′-((1-(4-(dimethylamino)phenyl)cyclopentyl)-methyl)urea or a salt thereof.

2. The method of claim 1, wherein effects on adrenocortical cells are assessed by measuring a reduction of cholesterol esters.

3. The method of claim 1, wherein effects on adrenocortical cells are assessed by measuring apoptosis.

4. The method of claim 1, wherein effects on adrenocortical cells are assessed by measuring a marker of apoptosis.

5. The method of claim 4, wherein the marker of apoptosis is Caspase 3, Caspase 7 or both Caspase 3 and Caspase 7 activity.

6. The method of claim 4, wherein the marker of apoptosis is activation of the unfolded protein response (UPR).

7. The method of claim 6, wherein activation of the UPR is determined by measuring expression of an activated XBP-1 splice variant.

8. The method of claim 1, wherein effects on adrenocortical cells are assessed by measuring a reduction of steroid biosynthesis.

9. The method of claim 8, wherein the steroid is cortisol.

10. The method of claim 1, wherein effects on adrenocortical cells are assessed by measuring cholesterol homeostasis.

11. A method for treating a disorder associated with aberrant adrenocortical cell behavior in a subject in need thereof, comprising administering to the subject a therapeutically effective amount of an agent which: (a) exhibits an IC₅₀ value against human ACAT1 (huACAT1) of less than 10 μM; and (b) disrupts cholesterol homeostasis in adrenocortical cells; wherein the agent is not N-(2,6-bis(1-methylethyl)-phenyl)-N′-((1-(4-(dimethylamino)phenyl)cyclopentyl)-methyl)urea or a salt thereof.

12. A method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) reduces steroid biosynthesis in adrenocortical cells, and wherein the agent is not N-(2,6-bis(1-methylethyl)phenyl)-N′-((1-(4-(dimethylamino)phenyl)-cyclopentyl)-methyl)urea or a salt thereof.

13. A method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) reduces mitochondrial function in adrenocortical cells, and wherein the agent is not N-(2,6-bis(1-methylethyl)phenyl)-N′-((1-(4-(dimethylamino)phenyl)-cyclopentyl)-methyl)urea or a salt thereof.

14. A method for treating a disorder associated with aberrant adrenocortical cell behavior in a subject in need thereof, comprising administering to the subject a therapeutically effective amount of an agent which: (a) exhibits an IC₅₀ value against huACAT1 of less than 10 μM; and (b) is preferentially bound by low-density lipoprotein (LDL), and wherein the agent is not N-(2,6-bis(1-methylethyl)phenyl)-N′-((1-(4-(dimethylamino)phenyl)-cyclopentyl)-methyl)urea or a salt thereof.

15. The method of claim 1, wherein the agent exhibits an IC₅₀ value against huACAT1 of less than 5 μM.

16. The method of claim 15, wherein the agent exhibits an IC₅₀ value against huACAT1 of less than 3 μM, 1 μM, 0.5 μM, 0.3 μM, 0.1 μM, 0.05 μM, 0.03 μM, 0.01 μM, 0.005 μM, 0.003 μM, or 0.001 μM.

17. The method of claim 1, wherein the agent is Compound No. 3 or Compound No. 5.

18. The method of claim 1, wherein the disorder is adrenocortical carcinoma, benign adenoma, increased hormone production, metastatic adrenocortical carcinoma, congenital adrenal hyperplasia, Cushing's syndrome, excess cortisol production, symptoms associated with excess cortisol production, hyperaldosteronism, 21-hydroxylase deficiency, reduces adrenocortical tumor size, or aberrant adrenal hormone production.

19. The method of claim 1, wherein the disorder is adrenocortical carcinoma.

20. The method of claim 1, wherein the disorder is Cushing's syndrome.
